(19) United States
(12) Patent Application Publication
(10) Pub. No.: US 2016/0235297 A1
(43) Pub. Date: Aug. 18, 2016

(54) MOLECULAR IMAGING METHODS FOR DIAGNOSIS AND EVALUATION OF OCULAR AND SYSTEMIC DISEASES

(71) Applicant: MASSACHUSETTS EYE AND EAR INFIRMARY, Boston, MA (US)

(72) Inventor: Ali Hafezi-Moghadam, Jamaica Plain, MA (US)

(21) Appl. No.: 14/880,536

(22) Filed: Oct. 12, 2015

Related U.S. Application Data
(63) Continuation of application No. 11/970,333, filed on Jan. 7, 2008, now abandoned.
(60) Provisional application No. 60/902,004, filed on Feb. 16, 2007.

(51) Int. Cl. A61B 3/12; A61B 5/145

(52) U.S. Cl. CPC A61B 3/1233; A61B 3/1241; A61B 5/14546

(57) ABSTRACT

This invention relates generally to minimally-invasive, in vivo methods of detecting one or more ligands on an intraluminal surface of a blood vessel using microparticles coated with one or more ligand binding partners. More particularly, in certain embodiments, the invention relates to minimally-invasive, in vivo methods of detecting endothelial and leukocyte antigens that are predictive of diabetic retinopathy (DR) and/or other conditions using protein-conjugated microparticles detectable by a non-invasive detection system, for example, a scanning laser ophthalmoscope. In other embodiments, the invention relates to targeted delivery of drugs or other substances to specific regions of an intraluminal surface of a blood vessel using drug-containing microparticles coated with one or more ligand binding partners.

### RELATED APPLICATION

This application claims priority to and the benefit of U.S. provisional patent application Ser. No. 60/902,004, filed Feb. 16, 2007, the disclosure of which is incorporated herein by reference in its entirety.

### GOVERNMENT RIGHTS

The invention was supported, in part, by grant A1050775 from the National Institute of Health (NIH). The Government has certain rights in the invention.

### FIELD OF THE INVENTION

This invention relates generally to minimally-invasive methods for evaluating and treating ocular and systemic diseases using microparticles. More particularly, in certain embodiments, the invention relates to in vivo methods of detecting one or more ligands on an intraluminal surface of a blood vessel by administering microparticles coated with one or more ligand binding partners and detecting the microparticles with a minimally-invasive detection system, for example, a scanning laser ophthalmoscope.

### BACKGROUND OF THE INVENTION

Diabetic retinopathy (DR) is a manifestation of diabetes in the microvasculature of the retina and is a leading cause of adult vision loss in the industrialized world. Some of the earliest clinically detectable changes are structural in nature, characterized by the occurrence of microaneurysms and small, dot-like hemorrhages (non-proliferative stage). Later, new vessels develop to compensate for the compromised circulatory system of the retina (proliferative stage). These new vessels hemorrhage easily, which impedes retinal function. Thus, clinically detectable signs of DR include small aneurysms, exudates, and new vessels in the eye.

Vision loss from DR may be preventable with early detection and treatment. However, many patients are diagnosed with DR after it is too late for an effective intervention. Those who present with a significantly reduced vision at the time of diagnosis have likely already substantial retinal injury, and the lost vision can rarely be restored.

The retinal and choroidal endothelium express specific surface antigens during the various stages of DR. By detecting these antigens and their patterns of expression in vivo, early signs of disease can be revealed. Early detection and staging of the disease prior to the manifestation of clinical symptoms would likely improve the chances for a successful therapeutic outcome.

Currently, there is no method that can detect retinal and choroidal endothelial antigens in vivo in humans or animals. There is also currently no method for sub-clinical molecular imaging of ocular vascular injury in viva. Current state-of-the-art fundus imaging techniques lack the resolution to visualize unlabeled leukocytes in retinal vessels. In an experimental setting, leukocytes can be visualized using invasive procedures (e.g., procedures involving cutting the cremaster muscle) and/or selective staining, e.g., through intravital microscopy or acridine orange fluorography (AOLF). However, neither of these techniques are possible with human patients.

Before the manifestation of observable structural damage to the retina in experimental DR, the retinal endothelium upregulates ICAM-1, causing leukocyte accumulation. ICAM-1 expression and leukocyte accumulation are sub-clinical signs of experimental DR, which coincide with endothelial dysfunction and vascular leakage. While early changes to the retinal endothelium can be studied in animals in an experimental setting, in diabetic patients there currently is no way to detect endothelial antigens or determine whether they play a role in the progression of DR. However, being able to identify such early markers in the clinic would provide greater opportunity to warn patients and initiate treatment at a stage that may offer a better chance to prevent loss of vision.

To improve the visual prognosis of diabetic patients, regular screening for treatable early signs of retinopathy is crucial. However, current limitations of ocular healthcare do not allow for regular screening. Only half of the patients with diabetes undergo an appropriate annual examination. The shortage of qualified specialists at many medical centers and the high costs involved result in an inadequate number of routine exams performed for appropriate screening.

Early diagnosis of ocular inflammation may prevent vision loss. There is a need for systems and methods for detecting signs of inflammation before the manifestation of clinical signs of disease or the occurrence of structural damage.

### SUMMARY OF THE INVENTION

The invention provides minimally-invasive methods for in vivo evaluation and molecular imaging of endothelial injury, for example, in the retinal and choroidal vessels of humans and live animals. The methods of the invention involve detecting one or more ligands on an intraluminal surface of a blood vessel, where the ligands are indicative of inflammation, disease, or other condition of the blood vessel. The invention uses microparticles with one or more substances coupled thereto, where the one or more substances interact and/or bind to the ligands. The interaction between the ligands and the binding partners on the microparticles affects the movement of the microparticles in the blood vessel. The movement of the microparticles can be detected using a non-invasive imaging device such as a scanning laser ophthalmoscope. Thus, the presence or absence of the ligands on the intraluminal surface of the blood vessel can be established, and any associated endothelial injury or condition can be assessed.

The methods can be used to diagnose subclinical signs of ocular inflammatory diseases, such as specific endothelial changes due to diabetic retinopathy, uveitis, or age-related macular degeneration (AMD). The methods may also be applied to the diagnosis and/or staging of other diseases with a vascular or inflammatory component, such as atherosclerosis, autoimmune diseases, or Alzheimer's Disease, as specific endothelial markers of these diseases are (or become) available.

The methods described herein can depict signs of DR at a much earlier stage than previously possible. For example, in certain embodiments, the methods allow in vivo detection of specific endothelial and leukocyte antigens that are predictive of DR, using protein-conjugated microparticles as fluorescent contrast agents. These antigens appear in early stages of DR, and they may be detected before clinical presentation of symptoms using methods described herein.

Much of the damage in DR is due to vascular changes, such as endothelial apoptosis and microvascular leakage. Leukocyte accumulation in the retinal vessels is causative of vascular leakage and endothelial damage and precodes clinical signs of DR. The vascular endothelium responds to pro-inflammatory signals with sequential presentation of specific surface antigens, such as P-selectin and ICAM-1. Similarly, firmly adhering leukocytes express surface antigens, i.e. CD18 and VLA-4. Methods presented herein can detect such endothelial surface antigens and leukocyte surface antigens in vivo.

The methods of the invention offer a powerful tool for non-invasive or minimally-invasive detection of DR-specific antigens. The methods can detect disease-specific molecular changes on the retinal and choroidal endothelium in vivo. Detection and diagnosis of sub-clinical signs of disease enables earlier therapeutic interventions. The methods are minimally-invasive—that is, in certain embodiments, only a one-time systemic injection of microparticles is performed. Furthermore, methods of the invention provide for the in vivo detection of ocular endothelial surface antigens in humans without either general or local anesthesia. By contrast, intravital microscopy of cremaster or mesentery requires deep anesthesia, due to the surgical procedures involved.

In certain embodiments, fluorescent microparticles conjugated with binding molecules are used to detect endothelial surface antigens in the retinal and choroidal vessels in human patients. Microparticles composed of albumin-shelled gas bubbles have been used in echocardiography to measure cardiac function, but conjugated microparticles have not been used in minimally-invasive, in-vivo detection methods for detection of native ligands on an intraluminal surface of a blood vessel. Other investigators have used endogenously labeled (e.g., with acridine orange) and exogenously labeled cells (e.g., from the spleen) to visualize leukocyte dynamics in the retinal vessels of mice with a scanning laser ophthalmoscope (SLO). However, such endogenously and exogenously labeled leukocyte visualization techniques are not clinically applicable for human patients.

The present approach using conjugated microparticles are clinically applicable for human patients. The methods derive from the surprising discovery that a scanning laser ophthalmoscope (and other devices of similar resolution) can be used to image rolling phenomena of fluorescent microparticles conjugated with binding molecules that bind to and/or interact with native ligands on the intraluminal surface of a blood vessel. Rolling and/or adhesion phenomena can be detected and quantified, and diagnostic and/or staging information derived therefrom.

SLO and similar non-invasive, in vivo systems have previously appeared to lack the resolution necessary for applications of this type. However, experimental results described herein show that rolling and/or adhesion phenomena can be detected and quantified using SLO to obtain an in vivo time-sequence of micrographs of one or more microparticles in an ocular blood vessel. An SLO device may have a resolution limit of about 5.7 micrometers per pixel, while microparticles of diameter of about 2 micrometers or less may be needed for a given application (e.g., to avoid clogging or plugging of capillaries). However, without wishing to be limited to any particular hypothesis, it is believed the diffusion and/or propagation of fluorescent light from the microparticle to the tissue makes the microparticle appear larger, and allows meaningful rolling and/or adhesion data to be obtained, for example, via SRO. Furthermore, optical detection is different from spatial visualization. For example, detection of a single photon may be possible with the unassisted eye in a dark room, while the spatial resolution of the eye (and of state-of-the-art imaging devices) is nowhere near the dimension of a photon.

In certain embodiments, two or more populations of microparticles may be coated with different binding molecules, wherein each population has distinct emission wavelengths. This allows side-by-side (e.g., simultaneous) examination of two or more endothelial antigens in the same blood vessel, and/or allows examination of the interaction of two or more substances in the blood vessel. Thus, multi-color SLO imaging may be performed.

In certain embodiments, the invention provides targeted drug (or other substance) delivery directly to an injured portion of a blood vessel using drug-carrying microparticles to which one or more ligand-binding substances are conjugated. The one or more binding substances bind to one or more ligands on a targeted intraluminal surface of the blood vessel as described in more detail herein above, thereby immobilizing the microparticles on the targeted intraluminal surface. Once the microparticles are immobilized, the release of the one or more agents from the microparticles onto the targeted intraluminal surface may be affected, for example, by administration of laser light (or any other electromagnetic radiation), a magnetic field, and/or a releasing agent. The release may also be affected by passage of time, where the microparticles break down over time allowing diffusion of the agent held within the microparticles onto/into the targeted region.

In certain embodiments, drugs or other substances are delivered to injured endothelium during acute or chronic inflammation, for example, uveitis, using markers of inflammation, such as selectins and their ligands, integrins and their ligands, etc. In other embodiments, drugs or other substances are delivered to injured endothelium during diabetic retinopathy or AMD using markers of neovascularizations, such as the αᵥβ₃ integrin.

In one aspect, the invention relates to a minimally invasive method for the in vivo detection of one or more ligands on an intraluminal surface of a blood vessel, the method including the steps of: (a) administering microparticles to a subject, the microparticles having an average diameter less than the diameter of a blood vessel of the subject in which the microparticles travel, and wherein the microparticles have a surface to which one or more substances are conjugated, wherein the one or more substances interact with one or more ligands on the intraluminal surface of the blood vessel, thereby inhibiting movement of the microparticles through the blood vessel; and (b) detecting one or more of the administered microparticles in the blood vessel using a non-invasive detection device. The one or more ligands may be native (endogenous) and/or exogenous. The one or more substances conjugated to the surface of the microparticles may be covalently bound to the surface of the microparticles or non-covalently associated with the surface. In certain embodiments, the microspheres are fluorescent microspheres and the detection device is a scanning laser ophthalmoscope. The description of elements of other aspects of the invention can be applied to this aspect of the invention as well.

The one or more ligands on the intraluminal surface may include one or more endothelial surface antigens and/or one or more leukocyte surface antigens. Alternatively or additionally, the one or more ligands may include one or more platelet antigens, cell surface molecules, micro-particle antigens, proteins, lipids, carbohydrates, glycoproteins, lipoproteins, bacterial antigens, viral antigens, parasite antigens, cancer cell antigens, and/or any combination thereof. The ligands may accumulate and/or become immobilized on the intraluminal surface of the blood vessel.

In certain embodiments, the method includes determining one or more rolling, tethering, and/or adhesion parameters of one or more of the microparticles in the blood vessel, wherein the one or more parameters are indicative of the presence of one or more of the ligands on the intraluminal surface of the blood vessel. The one or more ligands may include, for example, one or more endothelial surface antigens and/or one or more leukocyte surface antigens. The one or more parameters may be indicative of inflammation in the blood vessel.

The blood vessel is an ocular blood vessel in certain embodiments. For example, the blood vessel may be located in retinal tissue, choroidal tissue, iris tissue, or conjunctival tissue.

Where the blood vessel is an ocular blood vessel, for example, the method may be used to detect a sub-clinical manifestation of diabetic retinopathy based at least in part on the one or more microparticles detected in step (b), such manifestation being, for instance, an endothelial injury in a choroidal and/or retinal blood vessel. In certain embodiments, the method identifies (and/or locates) endothelial injury in choriocapillaris during endotoxin-induced uveitis.

In certain embodiments, the method detects a vascular change resulting from physiologic aging, based at least in part on the detected microparticles. The method may detect a change in permeability of a blood vessel based at least in part on the detected microparticles. The method may detect a change in the growth, degradation, and/or remodeling of a blood vessel.

Moreover, the method may further include diagnosing and/or staging a medical condition. The medical condition preferably has one or more vascular, inflammatory, immune, and/or thrombotic components—for example, the medical condition may be diabetic retinopathy, atherosclerosis, an autoimmune disease, Alzheimer's Disease, glaucoma, and macular degeneration (e.g. age-related macular degeneration, AMD). Other medical conditions that may be diagnosed or staged include neuronal or neuro-degenerative disease, thrombosis or hemostasis related disease, metabolic disease, vascular congenital disease, congenital disease, endocrine disease, trauma induced condition, hematological disease, oncological disease, renal or urological disease, hepatological disease, gastro-entrological disease, pulmonary disease, cardiac disease, manifestation of a therapeutic intervention, manifestation or side-effect of a pharmacological intervention, manifestation of substance abuse, genetic disease, nutritional or malnutritional disease, infectious disease, disease related to the extracellular matrix or connective tissues, and toxicological disease or condition related to toxic agents.

The microparticles may be rigid or elastic (or viscoelastic). In certain embodiments, the microparticles are fluorescent. The microparticles may be spherical, they may be (or include) shells, they may be lipid, polymer, and/or protein shells, they may be liquid droplets, and/or they may be filled or hollow. In certain embodiments, the microparticles are magnetic and/or paramagnetic. They may have a radiodensity greater than that of surrounding tissue (e.g. to facilitate radiodetection). In certain embodiments, the microparticles are filled and/or are made of a therapeutic substance (e.g., a drug) for targeted delivery.

The non-invasive detection device may include a scanning laser ophthalmoscope, a mydriatic retinal camera, a non-mydriatic retinal camera, a magnetic resonance imaging device, an ultrasound device, a computed tomography (CT) scanner, and/or an optical coherence tomography (OCT) device. The non-invasive detection device preferably detects one or more of the administered microparticles in vivo. Furthermore, the non-invasive detection device preferably detects one or more of the administered microparticles in the blood vessel in vivo, and/or without requiring breaking of, incision of, cutting of, and/or physical penetration of tissue of the subject, and/or without requiring surgical intervention. For example, in certain embodiments in which the blood vessel is ail ocular blood vessel, the non-invasive detection device detects one or more of the administered microparticles: in the blood vessel without requiring cutting a cremaster muscle of the subject.

In certain embodiments, the method includes administering two or more populations of microparticles, each population coated with different substances and having different emission and/or excitation wavelengths. For example, with the use of color scanning laser ophthalmoscopy (SLO), visualization of more than two populations is possible. In certain embodiments, the method includes identifying a change in growth, a degradation, and/or a remodeling of a blood vessel based at least in part on the one or mote microparticles detected.

The non-invasive detection device may capture a sequence of images over time to detect movement of one or more of the microparticles. The sequence of images may be used, for example, to determine a rolling velocity of one or more of the microparticles, thereby providing information about ligands on the intraluminal surface of the blood vessel.

The one or more substances conjugated to the surface of the microparticles in certain embodiments include one or more monoclonal antibodies, adhesion proteins, and/or peptides. The one or more substances conjugated to the surface of the microparticles may include one or more endothelial antigens, leukocyte antigens, platelet antigens, micro-particle antigens, bacterial antigens, viral antigens, parasite antigens, and/or cancer cell antigens. The one or more substances conjugated to the surface of the microparticles may include one or more substances accumulating on the intraluminal surface, for example, proteins, lipids, carbohydrates, glycoproteins, lipoproteins, and/or glycolipids.

In certain embodiments, the one or more substances conjugated to the surface of the microparticles include one or more selectins, integrins, immunoglobulins, cadherins, and/or lipoproteins. The substances conjugated to the surface of the microparticles may include, for example, one or more examples of one or more of the following: Selectins such as PSGL-1 or ESL-1; a selectin ligand; Integrins, such as CD18, VLA-4; Immunoglubulins, such as ICAM-1, or VCAM-1; Glycoproteins; Cadherins; Endothelial and epithelial junctional proteins; scavenger receptor, Tetraspanning membrane protein, also called transmembrane 4 (TM4); Sialyl-Lewis x (sLewis®) containing poly-N-acetyllactosamine Carbohydrate structures; complement and Complement control protein (CCP); Type II transmembrane glycoprotein; Mucins; TNF superfamily members; TNF receptors; cytokines; cytokine receptors; growth factors; growth factor receptors; chemokines; chemokine-receptors; G-protein coupled receptors; ADAMs; membrane-bound enzymes: Toll-like receptors (TLR); major histocompatibility complex family members; lectin superfamily members; Haemopoietin cytokine receptor superfamily members; members of insulin receptor family of tyrosine-protein kinases; EGFR family members; and/or Transferrin superfamily members.

In certain embodiments, the substances conjugated to the surface of the microparticles include CD18, Very Late Antigen-4 (VLA-4), and/or P-selectin Glycoprotein Ligand-1 (PSGL-1).

In these or other embodiments, the detecting step may include detecting one or more retinal and/or choroidal endothelial antigens such as P-selectin, Intercellular Adhesion Molecule-1 (ICAM-1), Vascular Cell Adhesion Molecule-1 (VCAM-1), P-selectin Glycoprotein Ligand-1 (PSGL-1), profiling, and/or desmoplakin, based at least in part on the detected microparticles.

In certain embodiments, the detecting step includes detecting one or more leukocyte antigens such as CD18 and VLA-4, based at least in part on the detected microparticles. In these and other embodiments, the method may include identifying one or more leukocyte antigens expressed by leukocytes that are firmly adhered to endothelium of diabetic retinal vessels, based at least in part on the one or more detected microparticles.

In certain embodiments, the microparticles have an average diameter of no greater than about 10 μm, no greater than about 7 μm, no greater than about no greater than about no greater than about 3 μm, no greater than about 2 μm, or no greater than about 1 μm, for example.

In certain embodiments, the subject is a vertebrate animal. In certain embodiments, the subject is a human.

In another aspect, the invention relates to a minimally invasive method for the in vivo determination of an endothelial condition associated with a disease, the method including the steps of: (a) administering fluorescent microparticles to a subject, wherein the microparticles have an average diameter less than a diameter of a blood vessel of the subject in which the microparticles travel, and wherein the microparticles have a surface to which one or more substances are conjugated, wherein the one or more substances are capable of interacting (e.g., binding) with an endothelial marker of the disease, thereby inhibiting movement of the microparticles through the blood vessel; and (b) detecting the administered fluorescent microspheres in one or more tissues of the subject using a scanning laser opthalmoscope. The description of elements of other aspects of the invention can be applied to this aspect of the invention as well.

In certain embodiments, the detecting step includes detecting a rolling, tethering, and/or adhesion parameter of one or more of the microparticles, which is indicative of the presence of one or more endothelial surface antigens and/or leukocyte surface antigens, particularly native antigens. These surface antigens may be indicative of a disease state, for example. In certain embodiments, the disease is diabetic retinopathy, however, in other embodiments, the disease may be atherosclerosis, an autoimmune disease, Alzheimer's Disease, glaucoma, or macular degeneration, for example.

In certain embodiments, the fluorescent microparticles have an average diameter of no greater than about 10 μm, no greater than about 7 μm, no greater than about 5 μm, no greater than about 4 μm, no greater than about 3 μm, no greater than about 2 μm, or no greater than about 1 μm, for example.

In yet another aspect, the invention relates to a method of detecting ocular inflammation in a subject, the method including: (a) administering fluorescent microparticles to a subject, wherein one or more substances are conjugated to the surface of the microparticles, wherein the one or more substances interact with (e.g., bind to) one or more endothelial surface antigens and/or leukocyte surface antigens (e.g., native antigens) located on an intraluminal surface of a blood vessel in the subject, thereby inhibiting movement of the microparticles through the blood vessel; and (b) determining a rolling, tethering, and/or adhesion parameter for one or more of the administered fluorescent microparticles in the blood vessel using a scanning laser opthalmoscope, wherein the one or more parameters are indicative of whether the subject has ocular inflammation. The description of elements of other aspects of the invention can be applied to this aspect of the invention as well.

In certain embodiments, the parameter indicates rolling of the fluorescent microparticles along an intraluminal surface of the blood vessel and/or adhesion of the fluorescent microparticles to an intraluminal surface of the blood vessel. The ocular inflammation may be indicative, for example, of diabetic retinopathy, age-related macular degeneration, and/or uveitis. In certain embodiments, the fluorescent microparticles have an average diameter of no greater than about 10 μm, no greater than about 7 μm, no greater than about 5 μm, no greater than about 4 μm, no greater than about 3 μm, no greater than about 2 μm, or no greater than about 1 μm, for example.

In still another aspect, the invention relates to a method for the delivery of one or more agents to a targeted intraluminal surface of a blood vessel, the method including: (a) administering to a subject microparticles carrying one or more agents, where the microparticles have an average diameter less than a diameter of a blood vessel of the subject in which the microparticles travel, and wherein the microparticles have a surface to which one or more binding substances are conjugated—the one or more binding substances bind to one or more ligands on a targeted intraluminal surface of the blood vessel, thereby immobilizing the microparticles on the targeted intraluminal surface; and (b) affecting the release of the one or more agents from the microparticles onto (e.g., includes “into”) the targeted intraluminal surface. The description of elements of other aspects of the invention can be applied to this aspect of the invention as well.

In certain embodiments, although the microparticles bind to the intraluminal surface, the released substance (e.g., drug) can diffuse to the vicinity (e.g., endothelium, vascular wall, and tissue surrounding the blood vessels) and the targeted region for microparticle binding may therefore be different from the targeted region for substance/drug delivery.

In certain embodiments, the administered microparticles carry the one or more agents in the interior of the microparticles, on the surface of the microparticles, and/or about (e.g., around and/or on) the microparticles.

The method may include applying electromagnetic radiation (e.g., non-invasively, from outside the body of the subject) to affect the release of the one or more agents from die microparticles onto the targeted intraluminal surface. In certain embodiments, the one or more agents are released by applying a magnetic field, ultrasound, and/or laser light. In certain embodiments, a releasing agent is administered, where the releasing agent affects the release of the one or more agents from the microparticles onto the targeted intraluminal surface. In certain embodiments, the step of affecting the release of the one or more agents comprises allowing sufficient time to pass such that the microparticles break down, thereby releasing the one or more agents (e.g., having been contained in the microparticles). Where the microparticle is itself made up of the agent, the step of affecting the release may comprise allowing sufficient time to pass such that the agent diffuses onto/into the targeted intraluminal surface.

The one or more agents may include one or more therapeutic agents, for example, autonomic drugs, cardiovascular-renal drugs, drugs affecting inflammation, drugs that act in the central nervous system, drugs for treatment of blood disease, drugs for treatment of inflammation, drugs for treatment of gout, drugs acting on blood, drugs acting on blood-forming organs, endocrine drugs, chemotherapeutic drugs, perinatal drugs, pediatric drugs, geriatric drugs, dermatologic drugs, drugs for treatment of gastrointestinal disease, botanicals, nutritional supplements, and/or homeopathic drugs. The one or more agents may include radio-isotopes, for example, for treatment of neoplasm (e.g., ocular melanoma or any other solid cancer).

In certain embodiments, the one or more substances conjugated to the surface of the microspheres includes a selectin, an integrin (e.g., avPs integrin), an immunoglobulin, a cadherin, and/or a lipoprotein. The one or more substances conjugated to the surface of the microspheres may include a marker of neovascularization, for example. In certain embodiments, the one or more ligands on the intraluminal surface include an endothelial surface antigen, a leukocyte surface antigen, or both.

In certain embodiments, the method delivers the one or more agents to injured endothelium during acute inflammation, chronic inflammation, uveitis, diabetic retinopathy, glaucoma, and/or macular degeneration (e.g., AMD).

In yet another aspect, the invention relates to a method for the delivery of one or more agents to a targeted intraluminal surface of a blood vessel, the method including the step of administering to a subject microparticles carrying one or more agents, where the microparticles have an average diameter less than a diameter of a blood vessel of the subject in which the microparticles travel, and wherein the microparticles have a surface to which one or more binding substances are conjugated—the one or more binding substances bind to one or more ligands on a targeted intraluminal surface of the blood vessel, thereby immobilizing the microparticles on the targeted intraluminal surface. The one or more agents may include radio-isotopes, for example, for treatment of neoplasm (e.g., ocular melanoma or any other solid cancer) located in, on, about, or in the vicinity of the blood vessel. The description of elements of other aspects of the invention can be applied to this aspect of the invention as well.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

While the invention is particularly shown and described herein with reference to specific examples and specific embodiments, it should be understood by those skilled in the art that various changes in form and detail may be made therein without departing from the spirit and scope of the invention.

FIGS. 1A-1D are schematic drawings illustrating the detection of endothelial surface antigens by using fluorescent microparticles conjugated with binding molecules mimicking leukocyte function, according to an illustrative embodiment of the invention.

FIGS. 2A and 2B are images demonstrating the in vivo visualization of leukocyte and microparticle rolling, according to an illustrative embodiment of the invention.

FIGS. 3A and 3B demonstrate quantification of L-selectin molecules on microparticles, according to an illustrative embodiment of the invention.

FIGS. 4A-4D demonstrate in vivo comparison of leukocyte and microparticle parameters, according to an illustrative embodiment of the invention.

FIGS. 5A-5C demonstrate that VLA-4 blockade significantly suppresses retinal leukostasis in diabetic animals, according to an illustrative embodiment of the invention.

FIGS. 6A-6E demonstrate acridine orange fluorography of endogenous leukocytes showing the role of VLA-4 in retinal leukostasis during DR, according to an illustrative embodiment of the invention.

FIGS. 7A-7D demonstrate visualization and a time course of platelet accumulation in choroidal vessels during EIU, according to an illustrative embodiment of the invention.

FIGS. 8A-8B demonstrate quantitative analysis of PSGL-1 conjugated to microparticles using flow cytometry, according to an illustrative embodiment of the invention.

FIGS. 9A-9C demonstrate the imaging of PSGL-1 conjugated fluorescent microparticles in choriocapillaris during acute inflammation, according to an illustrative embodiment of the invention.

FIGS. 10A-10C demonstrate accumulation of firmly adhering PSGL-1 conjugated microparticles in the choriocapillaris microcirculation of EIU animals, according to an illustrative embodiment of the invention.

FIGS. 11A-11D demonstrate the ex vivo visualization of the accumulation of PSGL-1-conjugated microparticles in the choriocapillaris and retinal vessels, according to an illustrative embodiment of the invention.

FIGS. 12A-12B demonstrate P-selectin mRNA-expression in choroidal vessels in EIU animals, according to an illustrative embodiment of the invention.

FIG. 13 is a block diagram depicting an scanning laser ophthalmoscope (SLO) system for use in certain embodiments of the methods described herein.

### DETAILED DESCRIPTION

It is contemplated that devices, systems, methods, and processes of the claimed invention encompass variations and adaptations developed using information from the embodiments described herein. Adaptation and/or modification of the devices, systems, methods, and processes described herein may be performed by those of ordinary skill in the relevant art.

Throughout the description, where apparatus, devices, and systems are described as having, including, or comprising specific components, or where processes and methods are described as having, including, or comprising specific steps, it is contemplated that, additionally, there are apparatus, devices, and systems of the present invention that consist essentially of, or consist of, the recited components, and that there are processes and methods according to the present invention that consist essentially of, or consist of, the recited processing steps.

It should be understood that the order of steps or order for performing certain methods is immaterial so long as the invention remains operable. Moreover, two or more steps or methods may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

The choriocapillaris is considered essential for the metabolic needs of the outer retina. Abnormalities of the choriocapillaris may compromise retinal function and lead to loss of vision, for instance in uveitis or central serous chorioretinopathy. In age-related macular degeneration (AMD) initial disturbance of the retinal pigment epithelium may lead to choroidal neovascularization. Early detection of choriocapillaris dysfunction may be important for initiating treatment at a time point that can prevent structural damage. In vivo visualization techniques of the choroidal microcirculation, including conventional fluorescein angiography or the experimental laser-targeted angiography for animals, have been used to investigate the choroidal vascular network and hemodynamic conditions. For example, using laser-targeted angiography, It is possible to detect fluorescein diffusion patterns in the choriocapillaris flow which reveal a lobelike structure in the choriocapillaris. However, these methods are not capable of evaluating leukocyte-endothelial interactions in choriocapillaris flow in vivo.

Leukocyte-endothelial interaction is fundamental to the pathogenesis of various ocular inflammatory diseases. At inflammatory sites endothelial cells express adhesion molecules that cause leukocyte recruitment in a multistep-process, which starts with rolling of leukocytes, continues with their firm adhesion, and may lead to transmigration into the extravascular space. Interacting leukocytes release cytokines, proteases, and reactive radical species, which contribute to the injury of the inflamed tissue. Leukocyte rolling, the first step in the recruitment process, is mediated primarily by the interaction between P-selectin on the endothelial surfaces and its main ligand, P-selectin glycoprotein ligand-1 (PSGL-1), constitutively expressed on the leukocyte surface. These specific biological processes that take place during inflammation may be used for non-invasive molecular imaging of ocular diseases.

Fluorescent microparticles conjugated with monoclonal antibodies (mAbs), peptides, or proteins that are known binding partners of specific endothelial antigens, such as ICAM-1 or P-selectin, interact with the retinal and choroidal endothelium in relation to the amount of endothelial injury that occurs, for example, during diabetes. Differences in microparticle-endothelial interaction patterns can be quantified using current clinical imaging devices, such as scanning laser ophthalmoscopy (SLO). Thus, fluorescent microparticles can serve as agents for the minimally-invasive detection and diagnosis of the early stages of DR.

Rolling and firm adhesion of microparticles can be monitored in the subject being investigated. If the microparticles exhibit such rolling and/or adhesion, the results indicate that the cells in the blood vessel are experiencing an adverse event, for example, an inflammatory response. These features can be tested in animal models. For example, in certain embodiments of the invention, rolling and adhesion parameters are measured in diabetic animals, untreated Wild-Type WT (negative control), and WT after treatment with proinflammatory mediators (positive control). Microparticles are conjugated to specific mAbs, adhesion proteins (i.e. PSGL-1 or L-selectin), or peptides known to interact with endothelial surface antigens in the retinal and choroidal vessels. Quantification of microparticle interactions in the retinal and choroidal vessels is performed after a brief and longer period of experimentally induced diabetes (2 and 10 weeks respectively) to track the progression of the DR. The targeted endothelial antigens can be semi-quantified at the mRNA or protein expression level in the retinal and choroidal tissues of the imaged animals. ConA stained retinal flat-mounts can be performed after in vivo microparticle injections to validate the outcomes of the microparticle adhesion to the retinal and choroidal vessels as imaged by SLO.

Furthermore, fluorescent microparticles conjugated to mAb, adhesion proteins, or peptides that are known binding partners of specific leukocyte antigens, such as CD18 and VLA-4, interact with accumulated leukocytes expressing these molecules when injected into the circulation. The fluorescent microparticles can be used to detect and quantify the amount of leukocytes that are known to accumulate in the retinal vessels during DR, through the binding of their CD18 to endothelial-ICAM-1.

Microparticle binding to firmly adhering leukocytes can be quantified and compared in the retinas and choroids of diabetic animals, untreated WT, and WT after treatment with proinflammatory mediators using SLO. ConA stained flat-mounts of the retinal and choroidal vessels can be performed to find out whether and to what percentage the microparticles targeting leukocyte antigens are bound to them.

Various methods described herein focus on events that occur prior to the manifestation of clinical signs of DR or visible structural damage to the retinal vessels. Within the first week of experimental diabetes, leukocytes adhere to and accumulate within the vasculature of the retina. The leukocyte increases coincide with the onset of diabetic vascular dysfunction. Initially, the leukocyte recruitment is moderate, however, it becomes more severe with time. Unfortunately, even state-of-the-art fundus imaging techniques currently lack the resolution to visualize unlabeled leukocytes in retinal vessels. Therefore, while it is possible after invasive procedures or selective staining to track leukocytes in an experimental setting, e.g., through intravital microscopy or acridine orange fluorography (AOLF), the same currently is not applicable to the clinical setting (e.g., because acridine orange is toxic in humans). Since certain imaging approaches described herein are based on molecular events that underlie this leukocyte recruitment, steps during leukocyte recruitment and the molecules generally involved are introduced herein, with the understanding that these molecules may have differential roles in leukocyte recruitment for different organ systems and specific disease states.

Leukocyte recruitment to sites of inflammation occurs in a cascade-like fashion. The endothelium sequentially expresses adhesion molecules and presents chemoattractants to the free flowing leukocytes to orchestrate the recruitment process. Leukocyte rolling, the initial step in the recruitment cascade, is followed by leukocyte activation, firm adhesion, and transmigration into the interstitial tissue. Various adhesion molecules, such as selectins, integrins, and immunoglobulins have roles in this process.

Selectins mainly mediate leukocyte tolling, the first step of leukocyte-endothelial interaction. P-selectin is the first adhesion receptor transiently upregulated on the endothelium during inflammation. P-selectin's binding to P-selectin-Glyco-Ligand-1 (PSGL-1) initiates leukocyte rolling. P-selectin is upregulated during ocular inflammation and DR. L-selectin is constitutively expressed on the leukocyte surface and enables through its interaction with endothelial ligands rolling of leukocytes on inflamed venules.

The activated endothelium expresses ICAM-1, which binds to leukocyte β₂ integrins (LFA-1 and Mac-1), mediating firm leukocyte adhesion. In experimental DR, leukocyte accumulation is largely due to ICAM-1 expression on the retinal endothelium. These experimental results correspond to a marked increase in ICAM-1 expression and leukocyte density in human eyes with DR, validating the clinical relevance of the experimental findings. In contrast, other investigators reported unaltered levels of ICAM-1 in diabetes. As described herein, other adhesion molecules, including vascular cell adhesion molecule-1 (VCAM-1) and its leukocyte ligand, very late antigen-4 (VLA-4), also play a significant role in the process of leukocyte recruitment during experimental DR. This is consistent with prior reports of increased VCAM-1 expression in experimentally-induced diabetes and increased levels of soluble VCAM-1 in the vitreous or plasma during the proliferative stage of diabetic retinopathy.

Experimental data indicate that the endothelial antigens are expressed even before the earliest clinical signs of DR, however, heretofore, there has been no method for their detection in human patients. Expression of these antigens coincides with the onset of diabetic vascular dysfunction, such as incipient breakdown of the blood-retinal barrier, premature endothelial cell injury and death, and capillary ischemia/reperfusion. While the endothelial injury of early DR may be reparable, upon progression, the vascular endothelium may no longer regenerate. With the loss of the capacity of the endothelium to compensate for cumulative injuries, irreversible retinal vascular dysfunction ensues. Therefore, to broaden the range of therapeutic options, methods presented herein utilize these sub-clinical signs of DR for much earlier staging and diagnosis.

Techniques for functional imaging of endothelial surface antigens upregulated during inflammation have been described in Hafezi-Moghadam et al., 2006. VLA-4 Blockade Suppresses Endotoxin-Induced Uveitis: In Vivo Evidence for Functional Integrin Upregulation. FASEB J. FASEBJ/2006/063909: published online Jan. 3, 2007 (pp. 1-11), incorporated herein by reference in its entirety. In this work, fluorescent microparticles were generated that contained a desired number of recombinant L-selectin molecules conjugated onto their surface to mimic the process of leukocyte recruitment. These microparticles then were injected into the circulation of a mouse and their interaction with the endothelium of the cremaster muscle was visualized by intravital microscopy. The L-selectin conjugated microparticles robustly mimicked leukocyte rolling on the inflamed endothelium, whereas they did not interact with non-inflamed endothelium. Thus, endothelial antigens can be functionally characterized and semi-quantified. Methods presented herein improve upon these methods and, in certain embodiments, provide a minimally-invasive molecular imaging technique for detection of antigens expressed on the injured endothelium and accumulated leukocytes during DR.

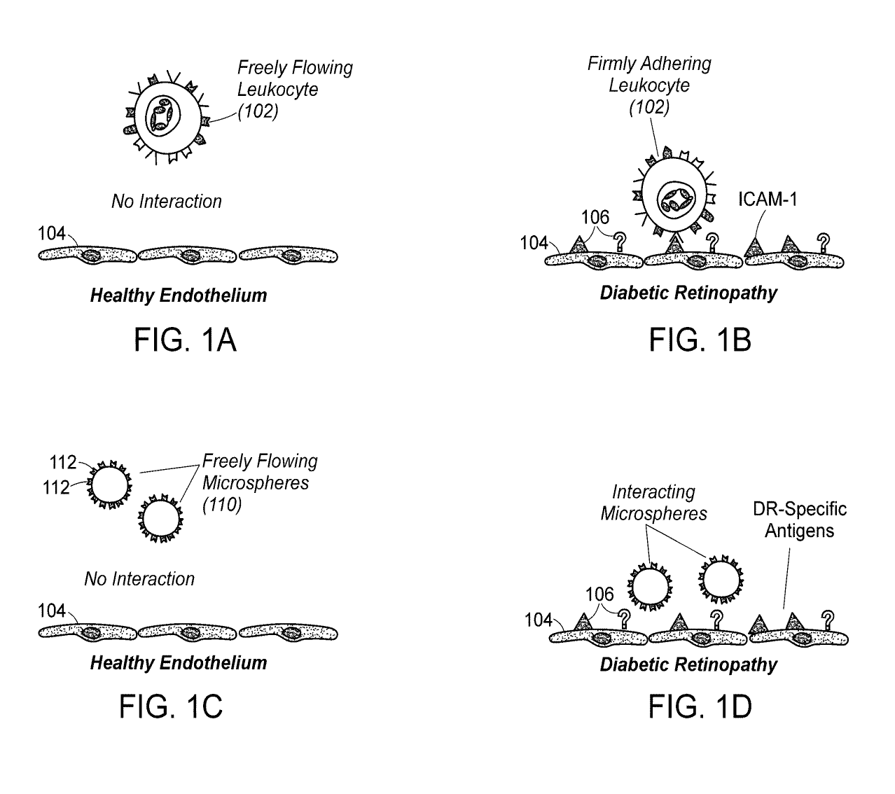

Inflammatory endothelial antigens may be detected by mimicking leukocyte recruitment. Because leukocyte recruitment precedes the clinical signs of DR, mimicking leukocyte recruitment with custom designed contrast agents that are more easily detectable than leukocytes, yet behave like miniature leukocytes and bind to the same endothelial surface antigens as leukocytes, provide the ability to visualize sub-clinical signs of DR (FIG. 1). Fluorescent microparticles emit a signal that is detectable in vivo, whereas unlabeled leukocytes currently remain obscured with existing fundus imaging techniques. Methods described herein detect fluorescent microparticles in the intact eye with existing clinical ophthalmic imaging techniques. Endothelial antigens (i.e. ICAM-1, VCAM-1, and P-selectin) may then serve as markers for DR diagnosis. An advantage that microparticles have compared to in vivo leukocyte staining techniques is the analytical specificity they allow, since they can be decorated with single molecules. The same amount of information cannot be easily gained from the interaction of leukocytes with the endothelium, as leukocytes express a variety of surface antigens with overlapping functions, making it difficult to pinpoint which molecule on the leukocyte surface is binding to the endothelium and therefore difficult to determine which endothelial antigen is involved.

FIGS. 1A-1D are schematic drawings illustrating the detection of endothelial surface antigens by using fluorescent microparticles conjugated with binding molecules mimicking leukocyte function. Under normal conditions, leukocytes 102 freely flow in the blood stream and do not interact with healthy endothelium 104 (FIG. 1A), except for occasional tethering. However, during diabetes, the endothelium 104 expresses endothelial antigens 106 that mediate firm leukocyte adhesion (FIG. 1B). The methods described herein use this principle to detect endothelial antigens in vivo. Fluorescent microparticles 110 having surfaces conjugated with specific binding partners 112 of endothelial antigens do not interact with the healthy endothelium 104 (FIG. 1C), unless the binding partners of their surface molecules are expressed on the endothelium 104 (FIG. 1D).

Another distinctive target for the molecular imaging methods described herein is firmly adhering leukocytes. Firmly adherent leukocytes express the β₂ integrin CD18, a specific marker of leukocyte activation. Furthermore, diabetic animals exhibit higher levels of surface integrin expression and integrin-mediated leukocyte adhesion, which allows the engineering of specific contrast agents to detect these leukocytes in the retinal vasculature.

Advantages of the methods described herein include the ability to detect and diagnose sub-clinical signs of DR, enabling earlier therapeutic interventions. Also, the methods are minimally-invasive, in that, for example, the only invasive aspect of the procedure is a one-time systemic injection of microparticles. Furthermore, the methods may be applied in the diagnosis and/or staging of other diseases with a vascular or inflammatory component, such as atherosclerosis, autoimmune diseases, or Alzheimer's Disease, where specific endothelial markers of these diseases exist or become available. Recent discoveries of disease specific endothelial changes, such as expression of the endothelial surface antigen, profilin, in diabetics indicate that this is a powerful strategy.

In certain embodiments, fluorescent microparticles are used to detect endothelial surface antigens in the retinal vessels. The use of similar agents in humans for different purposes has been long-term clinical practice. For instance, in contrast echo-cardiography, microparticles made of an albumin shelled gas bubble are used to measure cardiac function.

Evidence is presented herein that in vivo detection of endothelial surface antigens can be performed with high specificity by use of fluorescent microparticles to mimic aspects of leukocyte recruitment. Firstly, application of concepts of the invention are shown with respect to the murine cremasteric vasculature, with surgery and techniques utilizable in an experimental laboratory. Subsequently, it is shown that the same microparticle imaging approach is achievable using current state-of-the-art clinical ophthalmic devices, such as SLO, to detect retinal endothelial antigens during ocular inflammation in an intact eye.

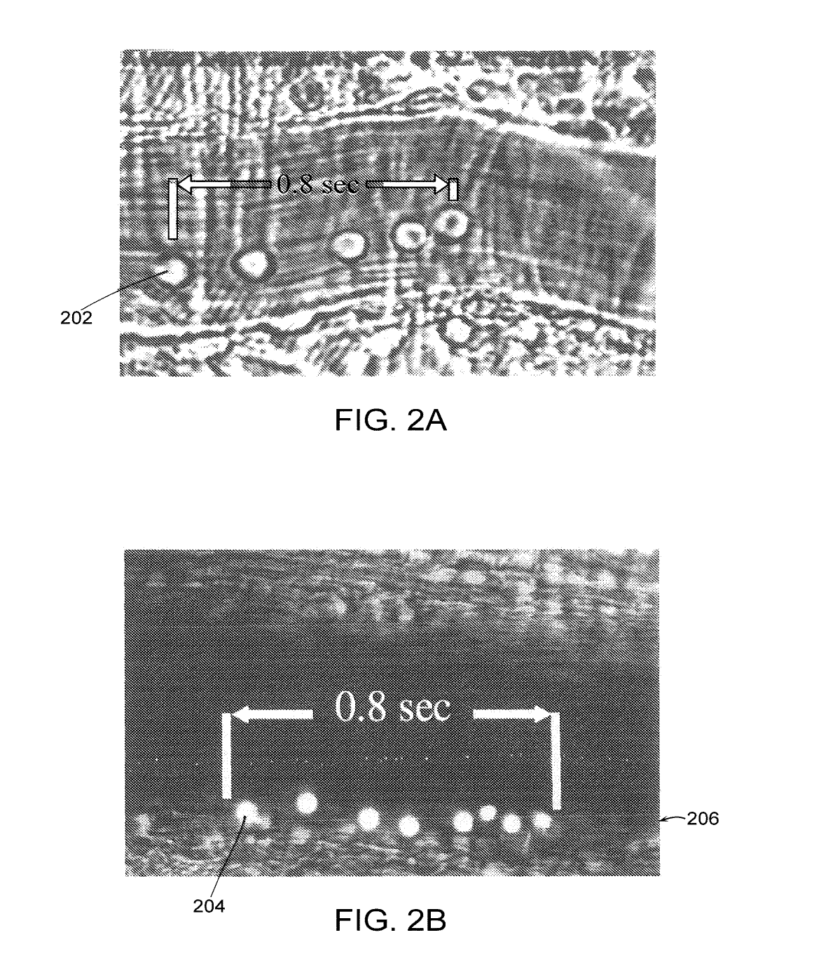

Endothelial surface antigens are detected in the cremasteric muscle using fluorescent microparticles. Experiments described herein functionally characterize endothelial antigens using an intravital microscopic method for tracking fluorescent microparticles in the murine cremaster vasculature. For this purpose, fluorescent microparticles were coupled to recombinant adhesion molecules (i.e. L-selectin), which are found on the surface of activated leukocytes. These microparticles in the circulation are shown herein to mimic specific aspects of leukocyte endothelial interaction, such as rolling and firm adhesion (FIGS. 2,3, & 4). This principle of using microparticles to mimic leukocytes can be used for detection of any endothelial surface antigen for which an interaction partner is available (i.e. protein, peptide, or mAb).

FIGS. 2A and 2B are images demonstrating the in vivo visualization of leukocyte and microparticle rolling. FIG. 2A is a composite image produced by superimposing a sequence of images obtained via transluminescence microcopy. FIG. 2A shows rolling of a representative leukocyte 202. FIG. 2B shows a similarly behaving L-selectin conjugated microparticle 204 on inflamed endothelium 206, as visualized via epifluorescence microscopy in the cremasteric microvessels of a live mouse. Superimposition of several video frames depicts the distances traveled in 0.8 s. Blood flows from left to right.

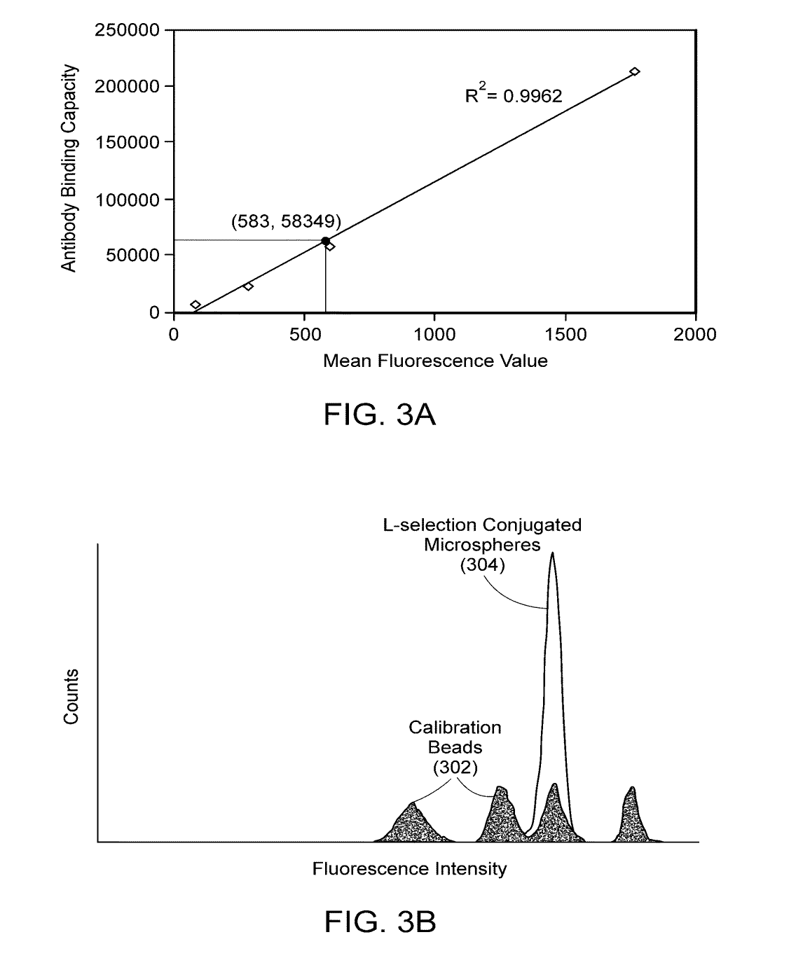

FIGS. 3A and 3B demonstrate a method of conjugating a desired number of molecules onto the surface of the microparticles to find an optimal number of copies for efficient rolling along the endothelial surface and/or firm adhesion. For instance, artificially high numbers of molecules on the microparticle surface may lead to their non-specific binding to the endothelium. To conjugate a desired number of molecules, various mixtures of two molecules were titrated, one that interacts with the endothelial antigens (e.g., L-selectin) and the second a non-interacting control (e.g., CD4), These molecules compete for the available binding sites on the surface of the microparticles and coat relative to their concentration ratios. It was confirmed by flow cytometry that the absolute number of L-selectin molecules per microparticle was 5.8×10⁴, similar to the number on leukocytes.

FIGS. 3A and 3B demonstrate quantification of L-selectin molecules on microparticles. Mean fluorescence values of calibration beads are shown in FIG. 3A and calibration beads 302 in flow cytometry are shown in the filled histograms in FIG. 3B. The fluorescence values of L-selectin conjugated microparticles 304 are shown in the open histogram in FIG. 3B. The number of L-selectin molecules conjugated to microparticles are based on the regression line in FIG. 3A.

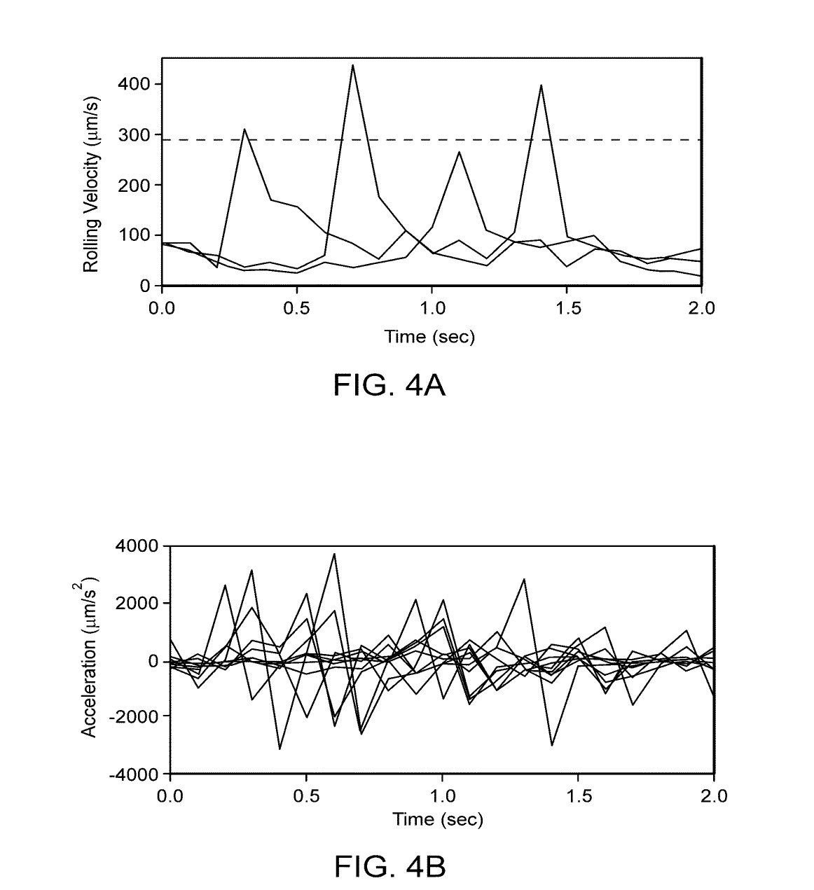

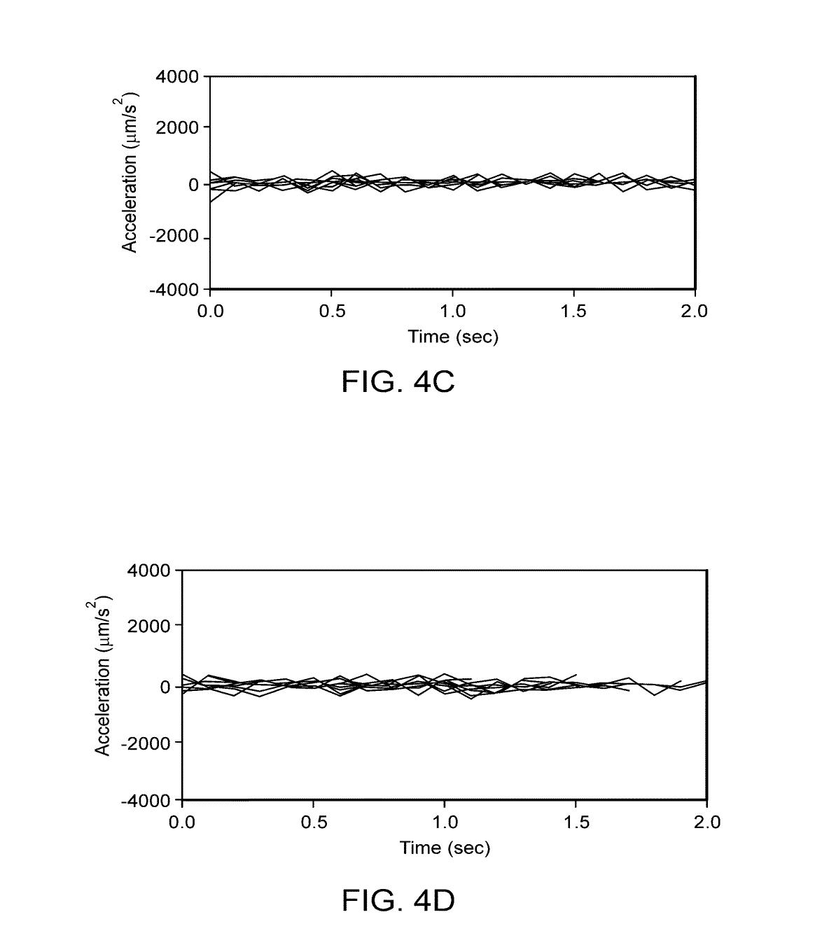

Rolling and adhesion parameters may be characterized. Different patterns of endothelial antigen expression lead to distinctive and quantifiable leukocyte tolling and Arm adhesion parameters. For example, leukocytes roll on cytokine treated vessels at slower velocities (5-10 μm/s) compared to the rolling velocities in less-inflamed vessels (i.e. 50 μm/s). Similarly, quantification of microparticle interaction parameters can reveal differences in endothelial antigen expression and injury. To demonstrate quantification of the microparticle interactions in vivo, L-selectin conjugated microparticles were injected into the cremaster muscle microcirculation of WT mice, intravital microscopy was performed. Comparison between microparticle- and leukocyte-interactions with the endothelium in the same vessels showed that the microparticles rolled with a low variability of velocity on TNF-α treated cremaster microvenules, similar to that of leukocytes (FIG. 4). This characteristic of the microparticles to mimic leukocyte rolling was used to judge the expression levels of inflammatory molecules on the endothelium, for instance during intravital microscopy.

FIGS. 4A-4D demonstrate in vivo comparison of leukocyte and microparticle parameters. Velocity profiles of three representative rolling leukocytes were measured at 0.1 sec intervals in an untreated WT mouse (FIG. 4A). The variability of rolling velocity of 10 random leukocytes over 0.1 sec intervals in untreated WT mice is shown in FIG. 4B, and in TNF-α-treated WT mice is shown in FIG. 4C. The variability of rolling velocity of 10 L-selectin conjugated microparticles on TNF-α-stimulated endothelium of WT mice is shown in FIG. 4D. These results show that rolling microparticles closely mimic the rolling profile of native leukocytes on the inflamed endothelium.

To show the specificity of the microparticle interaction in the cremasteric muscle, L-selectin conjugated microparticles were incubated with the neutralizing mAb, Mel-14, which significantly reduced the number of rolling microparticles from 23±5% to 4±1%; P<0.01. As a control, CD4 conjugated microparticles showed only 2±1% interaction with the endothelium.

The data presented above demonstrate that the microparticle imaging approach can be applied to the eye. The following experiments examine the feasibility of in vivo detection of retinal and choroidal endothelial antigens with fluorescent microparticles.

To investigate whether the inflammatory adhesion molecules, VLA-4 and VCAM-1, can serve as ocular imaging targets in the microparticle approach, their contribution to retinal leukostasis were observed in flat-mounts and in vivo.

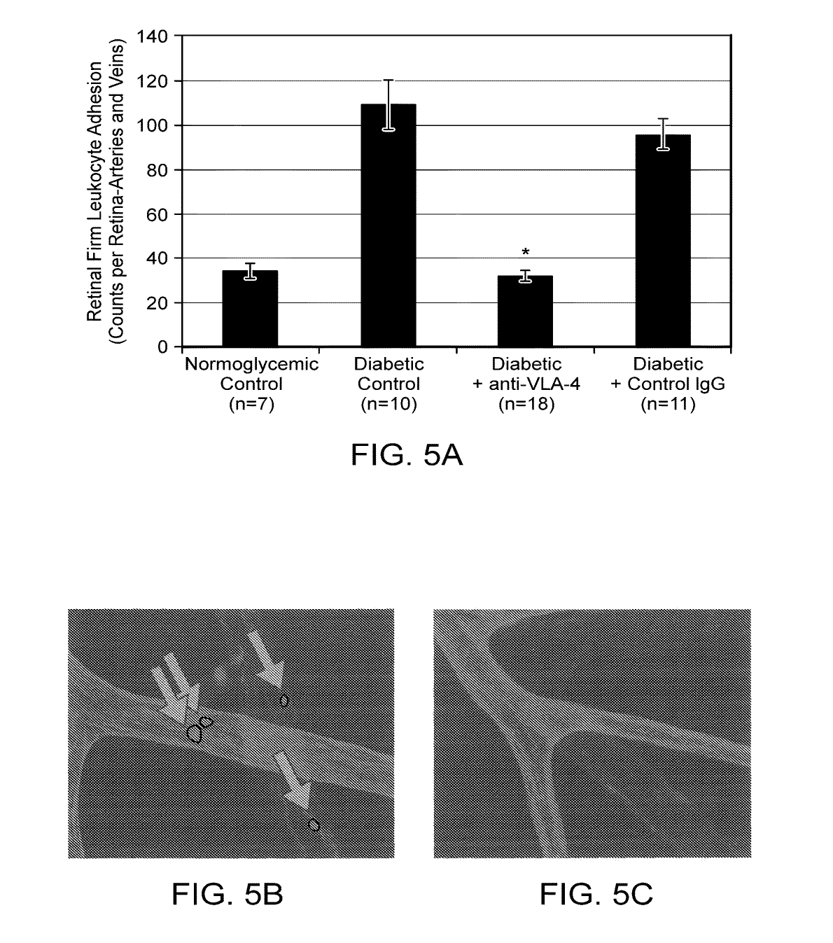

Experiments were conducted to demonstrate VLA-4/VCAM-1 mediated Firm leukocyte adhesion to retinal vessels during diabetes. Retinal leukocyte adhesion was quantified in normal and diabetic rats and significantly increased firm leukocyte adhesion was found in the diabetic animals (P<0.001) (FIG. 5). To investigate the role of endothelial VCAM-1 and leukocyte VLA-4 in this process and test whether these molecules may be suitable targets for molecular imaging, diabetic rats were treated with 1 mg/kg/day of a functional VLA-4 blocking antibody (clone TA-2, mouse IgG1, Seikagaku America, Cape Cod: lot #307-13-3-8) intraperitoneally. Two weeks after diabetes induction, anti-VLA-4 treatment drastically reduced the amount of retinal leukostasis, while an isotype-matched control mAb did not assert any significant reduction in firm leukocyte adhesion (FIG. 5). These data suggest the finding that VLA-4 interaction with endothelial VCAM-1 is an important early event in diabetic retinopathy and thus can be used for molecular detection.

FIGS. 5A-5C demonstrate that VLA-4 blockade significantly suppresses retinal leukostasis in diabetic animals. FIG. 5A is a graph showing average numbers of firm leukocyte adhesions in retinas of normal and diabetic rats, treated with anti-VLA-4 mAb (1 mg/kg) or control IgG by i.p. injection, p<0.001. FIGS. 5B and C are micrographs representing retinal vessels of diabetic rats after ConA-staining. FIG. 5B represents vehicle treated and FIG. 5C represents VLA-4 blockade. Arrows in Figure SB point to firmly adhering leukocytes.

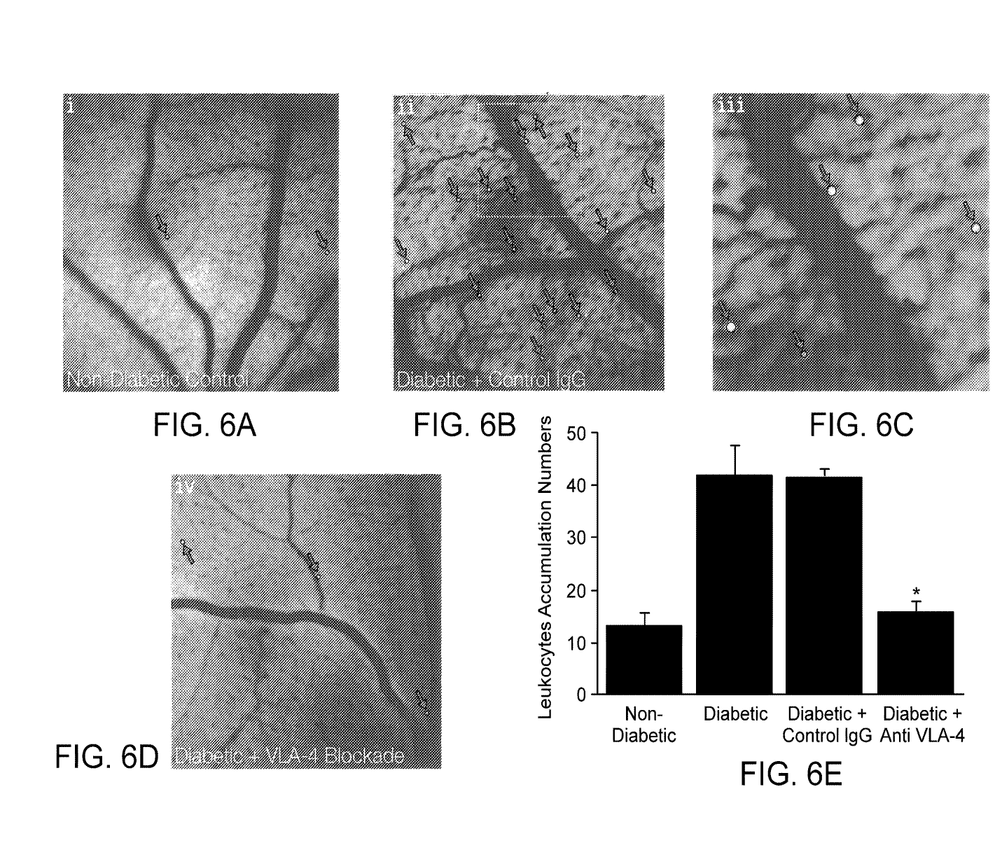

Experiments were performed to demonstrate VLA-4/VCAM-1 mediated diabetic leukocyte recruitment in vivo. To further investigate the role of VLA-4 in diabetic leukocyte recruitment in the retinal microcirculation, Scanning Laser Ophthalmoscopy (SLO) was used in combination with acridine-orange staining (AOLF) of the endogenous peripheral blood leukocytes (FIG. 6). The number of firmly adhering leukocytes in the retinal vessels of normal and diabetic Long Evans rats with and without the use of a VLA-4 neutralizing antibody or control IgG were quantified. Significantly higher leukostasis was found in the retinas of diabetic animals (36.7±6.1) compared to normal animals (13±2.4, P<0.01) (FIG. 6). When animals were treated with the neutralizing mAb, the number of firm adhesions was significantly reduced (14.2±1.2, P<0.01) compared to the diabetic animals or diabetic animals that received the same amount of a control IgG (41.2±1, P=0.5). These in vivo measurements confirm the ConA data shown above and demonstrate important role of VLA-4/VCAM-1 interaction in ocular inflammation. The in vivo (FIG. 6) and ex viva data (FIG. 5) indicate that VLA-4 and VCAM-1 serve as novel molecular imaging targets for detection of ocular inflammation.

FIGS. 6A-6E demonstrate acridine orange fluorography of endogenous leukocytes showing the role of VLA-4 in retinal leukostasis during DR. FIGS. 6A-6D show SLO images from retinas of normal (FIG. 6A) and diabetic Long Evans rats with (FIG. 6D) and without (FIGS. 6B and 6C) blockade of VLA-4.30 min after systemic acridine orange injection to slain endogenous leukocytes. The micrograph in FIG. 6C magnifies the area 610 outlined in FIG. 6B. Arrows indicate individual leukocytes accumulated in the retinal vessels. FIG. 6E shows retinal leukocyte accumulation in normal and diabetic animals, treated with a VLA-4 blocking mAb, control IgG, or vehicle alone (n=6 in each group). Data represent average values per retina±SEM. “*” indicates p<0.01.

Using Acridine Orange Leukocyte Fluorography (AOLF), it is possible to observe leukocyte recruitment in the retinal microcirculation to study the role of specific antigens in ocular diseases (FIG. 6). However, up to now it has not been possible to observe cellular interactions in the choriocapillaris with this technique. This is partly because the fluorescence signal of AO-labeled cells is not strong enough to distinguish itself from the background AO-staining of the endothelium and the various layers of the retinal and choroidal circulation. In order to examine cellular interactions in the choriocapillaris by SLO, it is necessary first to isolate the cells, for instance leukocytes or platelets, exogenously label them with a fluorescent dye, and inject them into a live animal. This procedure eliminates the background staining of, for instance, endothelial cells and improves the signal to noise ratio.

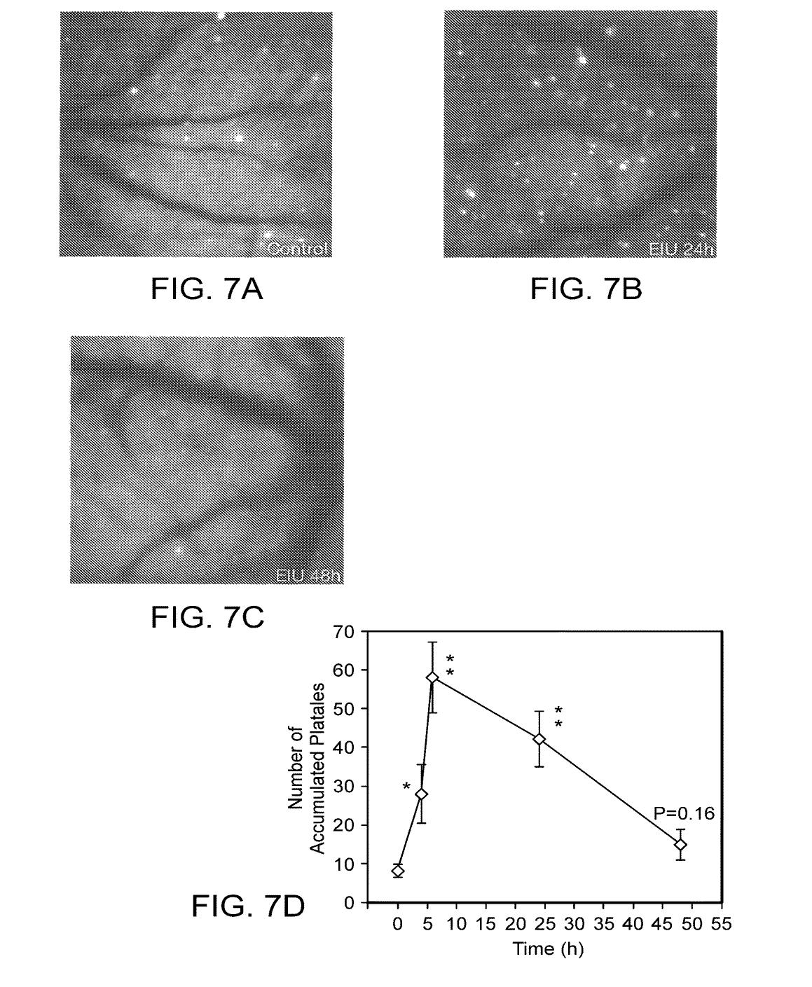

Labeled Platelets can be used to detect choroidal endothelial injury during EIU. To visualize cellular interactions with the choroidal vascular endothelium during Endotoxin-induced Uveitis (EIU), an established model of acute retinal inflammation, platelets were harvested from normal donor rats and stained them with carboxyfluorescein diacetate succinimidyl ester (Sigma Chemical, St. Louis, Mo.). Then 6×10⁸ fluorescently labeled platelets were infused into congenic recipients through a tail vein catheter at different time points after EIU, and visualized the retina using the HRA2 SLO. Platelet-endothelial interaction in the choroidal microcirculation was evaluated at 0, 4, 6, 24, and 48 hours after LPS injection with an SLO and recorded for further analysis. By changing the depth of focus on the SLO-device, it is possible to distinguish cellular interactions in the retina from those in the choroid. Platelet-endothelial interactions were quantified 30 min after the systemic injections. A significantly higher number of platelets accumulated in the choroid of EIU animals compared to normal animals, with a peak of accumulation 6 hrs after EIU induction (FIG. 7). A similar course of events was observed in the retinal vessels of these animals. These findings show that exogenously labeled platelets can be used to visualize and quantify the extent of choroidal endothelial injury in vivo. However, since platelet recruitment is mediated by a variety of antigens (including PSGL-1, P-selectin, gpIb, II_{b}IIₐ), the increased accumulation does not reveal the isolated increase of a specific endothelial antigen.

FIGS. 7A-7D demonstrates visualization and time course of platelet accumulation in choroidal vessels during EIU. Rat platelets were isolated, fluorescently labeled, and injected at different time points into EIU animals through a tail vein catheter. FIGS. 7A, 7B, and 7C show SLO still frames at a view angle of 30° and 15 frames per sec. White dots represent accumulated platelets in choroidal vessels of normal and EIU rats. FIG. 7D shows quantitative analysis of platelet accumulation in choroidal vessels of normal (0 h) and EIU rats at 4, 6, 24, and 48 h after LPS injection. Mean±SEM, n=4 animals per group. P<0.05 and P<0.01, compared with control.

Experiments were conducted to show that conjugated microparticles, when in the circulation, behave similarly to the animal's own leukocytes or the injected platelets and interact with specific antigens of the endothelium, revealing the amount of injury during a disease state. Since the microparticles emit a stronger fluorescent signal than endogenously labeled leukocytes or platelets and, similar to exogenously labeled cells, have a better signal to noise ratio achievable than in AOLF, it is possible to detect their interaction not only in the retinal, but also in the choroidal vessels of non-pigmented animals. The superior signal-to-noise ratio of the microparticles is partly a result of not using soluble dyes (i.e. acridine orange) that would nonspecifically stain a variety of cells, such as the endothelium. Because current in vivo methods for evaluation of the choriocapillaris have limitations, it is now possible to detect the effects of diabetes-induced inflammation on the choriocapillaris.

To target retinal and choroidal endothelial antigens, leukocyte and/or platelet adhesion molecules were conjugated to the surface of microparticles and their recruitment behavior was investigated in vivo.

Further experiments were performed to demonstrate non-invasive visualization of endothelial injury during ocular inflammation under physiologic flow conditions. To accomplish this, PSGL-1-conjugated fluorescent microparticles (microspheres) were generated with known site-densities and their interaction with the vascular endothelium of die choriocapillaris was detected and quantified using SLO. The PSGL-1 molecule we used, rPSGL-Ig, is a fully human recombinant fusion protein of PSGL-1 and IgG-1 that has been in clinical trials for acute myocardial infarction and renal and liver (personal communication) transplant for prevention of ischemia reperfusion injury. Since the molecule is tolerated in humans, the imaging technique may thus be used for early diagnosis of human ocular inflammatory diseases.

All experiments were performed in accordance with the ARVO Statement for the Use of Animals in Ophthalmic and Vision Research and were approved by the Animal Care and Use Committee of the Massachusetts Eye & Ear Infirmary. Male Lewis rats (8-10 weeks old; n=84) were obtained from Charles River (Wilmington, Mass.). Uveitis was induced in rats by injecting 100 μg of lipopolysaccharide (LPS; Salmonella typhimurium; Sigma Chemical, St. Louis, Mo.) diluted in 0.1 ml sterile saline into one hind footpad of each animal. Control animals received a footpad injection of saline alone. All rats were maintained in an air-conditioned room with a 12-hour light/dark cycle and were given free access to water and food until used for the experiments.

Carboxylated fluorescent or non-fluorescent microparticles (2 μm, Polysciences, Inc.; Warrington, Pa.) were covalently conjugated to protein G (Sigma) using a carbodiimide-coupling kit (Polysciences, Inc.). Recombinant P-selectin glycoprotein ligand-Ig (rPSGL-Ig: Y's Therapeutics, Inc.; Burlingame, Calif.) was incubated with the microparticles at 0.4 mg/ml overnight at room temperature. Microparticles were washed in PBS with 1% BSA before use in vivo. 6×10⁸ fluorescent microparticles were injected in each rat.

The average number of PSGL-1 molecules on the microparticle surfaces were determined using flow cytometry as previously described. Non-fluorescent microparticles (10⁶/ml, Polysciences, Inc.; Warrington, Pa.) conjugated to PSGL-Ig (Y's Therapeutics, Burlingame, Calif.) were incubated with PE-conjugated mouse anti human PSGL-1 (KPL-1) or its isotype-matched control (BD Biosciences, Franklin Lakes, N.J.) for 30 min, centrifuged at 4000 G for 5 min. washed twice and resuspended into PBS. The fluorescence intensity of 10⁴ microparticles was measured on a FACScan (Coulter EPICS XL), equipped with the ‘System Work II’ software. The surface expression was presented as the mean channel fluorescence on a logarithmic scale.

In parallel, calibration beads (Quantum Simply Cellular, Bangs Laboratories, Fishers, Ind.) were coated with reference fluorescence antibodies, as previously described. Four different populations of microparticles with known densities of binding sites for Fc were coated with goat anti-mouse IgG. Uncoated microparticles were used as control. A calibration curve was constructed based on the mean fluorescence intensity of the microparticles, using the quickcal software (V2.3, Bangs Laboratories, Fishers, Ind.).

To evaluate microparticle rolling in the rat choriocapillaris during EIU, a scanning laser ophthalmoscope was used (SLO, HRA2; Heidelberg Engineering, Dossenheim, Germany), coupled with a computer-assisted image analysis system to make continuous high-resolution images of the fundus. An argon blue laser was used as the illumination source, with a regular emission filter for fluorescein angiography, since the microparticle's spectral properties are comparable with those of sodium fluorescein. The images were obtained at a rate of 15 frames/s and recorded on a computer for further analysis (11). The experiments were performed at 4, 10, 24, 36, and 48 h after LPS injection. Six rats were used at each time point.

Immediately before microparticle injection, the rats were anesthetized with xylazine hydrochloride (10 mg/kg) and ketamine hydrochloride (50 mg/kg), and their pupils were dilated with 0.5% tropicamide and 2.5% phenylephrine hydrochloride. A contact lens was used to retain corneal clarity throughout the experiment. A catheter (BD Insyte™ Autoguard, 24GA, Ref #381412) was inserted into the tail vein of each animal. Animals were placed on a platform, allowing flexible positioning of the animals in relation to the SLO. Microparticles (6×10⁸/ml in saline) were injected continuously through the catheter for 1 min at a rate of 1 ml/min. Rolling microparticles were defined as microparticles that moved at a velocity significantly lower than that of free-flowing microparticles. The number of rolling microparticles was obtained from 30 seconds of the recordings.

Thirty minutes after microparticle injection, the fundus was imaged by SLO for quantification of the accumulated microparticles in the choriocapillaris. The number of fluorescent dots in the temporal (frame temporally next to the optic disk) and central area (frame with the optic disk in the center) of the choriocapillaris was counted.

To prepare retinal and choroidal flatmounts, animals were anesthetized 4 h after LPS injection. Subsequently, microparticles (6×10⁸/ml in saline) were injected continuously through the tail vein catheter for 1 min at a rate of 1 ml/min. Thirty minutes after microparticle injection, animals were perfused with rhodamine-labeled concanavalin A lectin (Con-A; Vector Laboratories), 10 μg/mL in phosphate buffered saline ([PBS], pH7.4) to stain vascular endothelial cells and firmly adhering leukocytes. Perfusion was performed after the chest cavity was opened and a 24-gauge needle was introduced into the aorta. Drainage was achieved by opening the right atrium. Hie animals were then perfused with 20 mL PBS containing 2% paraformaldehyde to wash out intravascular content and unbound microspheres. Immediately after perfusion, the retina and choroid were microdissected and flatmounted, using a fluorescence anti-fading medium (Vector Laboratories).

The tissues were then observed under an epifluorescence microscope (DM RXA; Leica, Deerfield, Ill.), with both a FITC filter (excitation, 488 nm; detection, 505-530 nm) and a rhodamine filter (excitation, 543 nm; detection, >560 nm). Images were obtained using a high sensitivity digital camera, connected to a computer-assisted image analysis system. Using the openlab image analysis software, merged images of the microparticles (green fluorescent dots) with the retinal and the choroidal tissues (red) were generated.

Total RNA was isolated from the RPE-Bruch's membrane-choroid complex after removal of the neural retina using TRIzol reagent (Invitrogen; Carlsbad, Ca). The extracted RNA was quantified, and 1 μg of the RNA was used to make cDNA with First-Strand cDNA Synthesis Kit (Amersham Biosciences; Piscataway, N.J.). For semiquantitative PCR, 1 μl of each first-strand reaction was then amplified using P-selectin- and GAPDH-specific oligonucleotide primers. PCR amplification was performed with denaturation at 94° C. for 1 min, annealing at 55° C. for 1 min, and polymerization at 72° C. for 1 min. The reaction was performed for 35 cycles for P-selectin and 25 cycles for GAPDH. The primers were CAAGAGGAACAACCAGGACT (sense) and AATGGCTTCACAGGTTGGCA (anti-sense) for P-selectin, and TGGCACAGTCAAGGCTGAGA (sense) and CTTCTGAGTGGCAGTGATGG (anti-sense) for GAPDH. After completion, the reactions (6 μl) were analyzed by agarose gel electrophoresis and ethidium bromide staining (11).

All values are expressed as mean±SEM. Data were analyzed by Student's t-test. Differences between the experimental groups were considered statistically significant or highly significant when the probability values were <0.05 or <0.01, respectively.

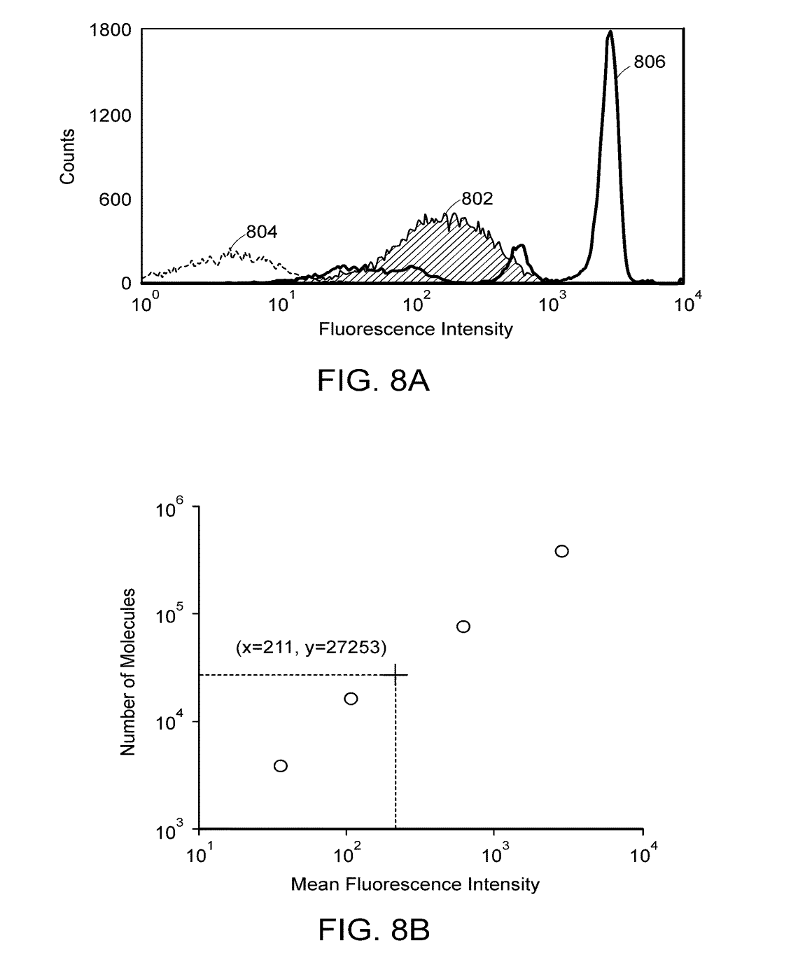

To quantify the number of PSGL-1 molecules conjugated onto the surface of our microparticles, non-fluorescent carboxylated microparticles covalently bound to protein G were generated and subsequently coated with recombinant PSGL-1. A PE-conjugated anti-PSGL-1 mAb or its isotype-matched control was used to label the PSGL-1 on the microparticles and their fluorescent intensities were measured by flow-cytometry (FIG. 8A). FIG. 8A is a flow cytometric histogram of PSGL-1-conjugated microparticles 802 (diagonal lines) labeled with PE-conjugated anti-PSGL-1 mAb. isotype control 804 (dotted line), and calibration beads with known binding sites 806 (solid line). Microparticles conjugated with PSGL-1 showed a mean fluorescence of 211.5, when incubated with PE-conjugated anti-PSGL-1 mAb, compared with 7.1, when incubated with isotype-matched control (FIG. 8A). To convert the mean fluorescence intensity obtained from the PSGL-1-conjugated microparticles to specific numbers of PSGL-1 molecules, the fluorescence intensities of calibration microbeads with known site densities of PE-conjugated IgG were acquired, and were examined under the same flow-cytometric setting (FIG. 8A). From the mean fluorescent intensities of the microbeads, a calibration curve was generated (R²=0.9997), indicating that in average 27,253 PSGL-1 molecules were bound on the surfaces of our microparticles (FIG. 8B). FIG. 8B depicts flow cytometric quantification of mean fluorescence values of calibration beads (◯) after incubation with PE-conjugated IgG. The mean fluorescence value of PSGL-1-conjugated microparticles is depicted as (±). The calculated copy number of PE-KPL-1 bound to PSGL-1-conjugated microparticles is based on linear regression (y=136x−1533, R²=0.9997).

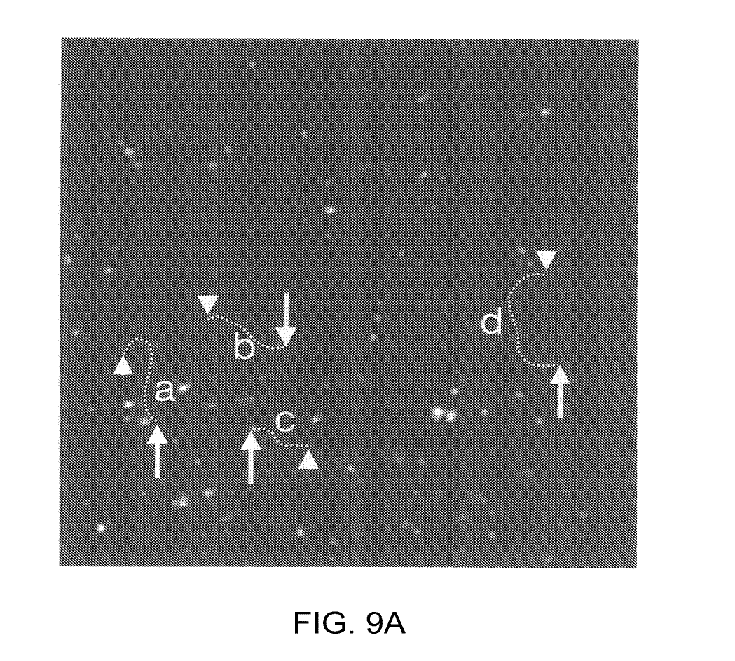

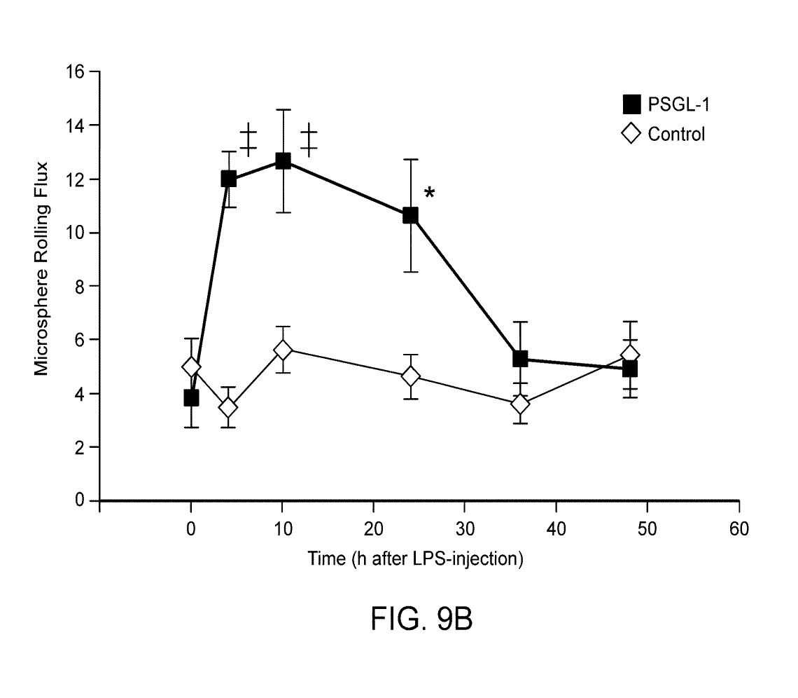

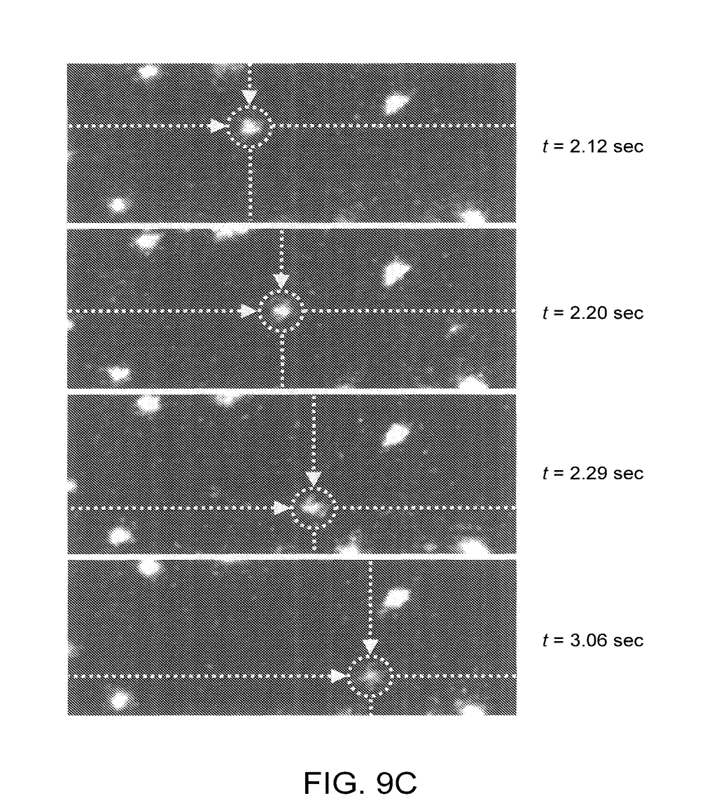

Immediately after intravenous injection of the PSGL-1-conjugated microparticles, free flowing and rolling microparticles were observed in the choriocapillaris of the examined rats (FIG. 9A). FIG. 9A depicts the movement of PSGL-1-conjugated fluorescent microparticles as detected in the choriocapillaris flow 4 h after LPS injection. Tracks of rolling microparticles are shown as white lines. Rolling microparticles moved within small limited areas, corresponding to the previously described lobules (10). White arrows indicate the points of appearance of individual rolling microparticles, while arrowheads show the points of disappearance of the same rolling microparticles. Other white spots indicate non-interacting, freely flowing microparticles.

FIG. 9B shows rolling flux of control and PSGL-1-conjugated microparticles in the choriocapillaris of normal (0 h) and EIU rats at different time points (4, 10, 24, 36, and 48 h) after LPS injection. Values are expressed as mean±SEM, n=6 animals in each group, * p<0.05, ‡ p<0.01. In the untreated control group (time 0), only very few PSGL-1-conjugated microparticles showed rolling interaction with the endothelium of the choriocapillaris (3.8±1.1) (FIG. 9B). However, 4 and 10 h after LPS injection, the number of microparticles rolling along the venous walls increased significantly (12±1, p=0.0003 at 4 h and 12.7±1.9, p=0.03 at 10 h), suggesting an increase in endothelial P-selectin expression at these time points (FIG. 9B). Twenty-four hours after LPS injection, the flux of rolling microparticles, although still significantly elevated, started to decline (10.7±2.1, p=0.016). This decline continued 36 and 48 h after LPS injection (5.3±1.4, p=0.4 and 5±1.1, p=0.5, respectively), suggesting a resolution of the acute inflammatory reaction (FIG. 9B). To illustrate the rolling of microparticles in the choriocapillaris, a representative PSGL-1-conjugated microparticle is followed by freeze frame advancing while the elapsed tracking time is indicated (16,23) (FIG. 9C). FIG. 9C depicts a sequence of fundus images 4 h after LPS injection, showing displacement of a rolling PSGL-1-conjugated fluorescent microparticle in the choriocapillaris of an EIU rat, where t is elapsed time after administration of fluorescent microparticles.

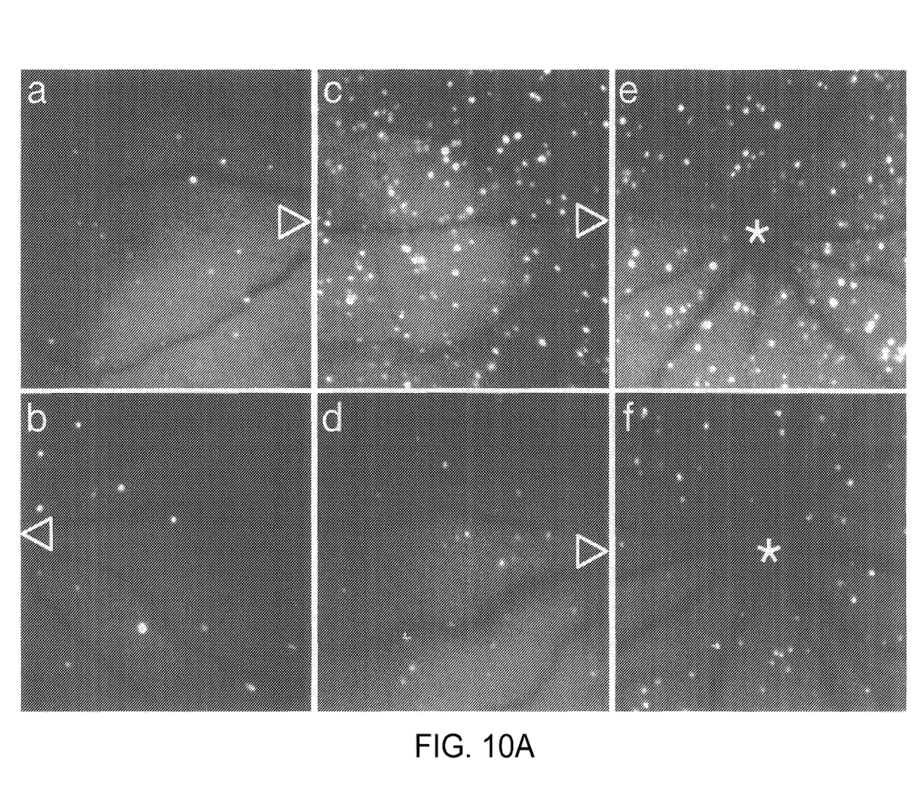

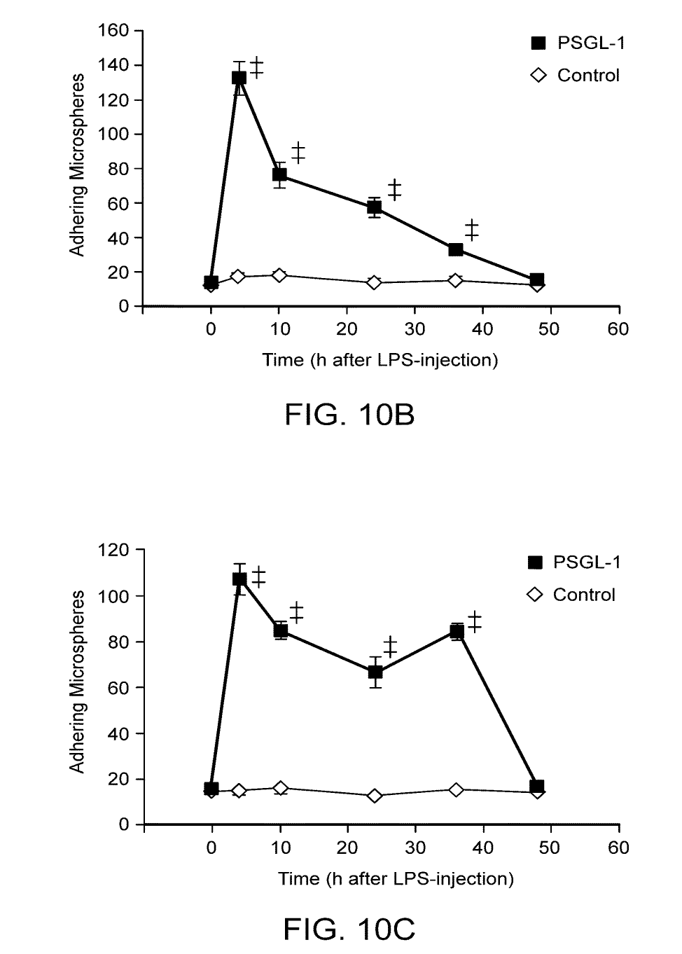

Thirty minutes after the initial injection of the conjugated microparticles, the number of free-flowing microparticles in the choriocapillaris of normal and EIU rats was substantially diminished, presumably due to the interaction of the microparticles with the endothelium of the vessels throughout the body. This allowed identification and quantification of the number of accumulated microparticles in the choriocapillaris as distinct stationary fluorescent marks with very high contrast against the non-fluorescent background (FIG. 10A-C). Microparticle accumulation in the choriocapillaris was investigated using a scanning laser ophthalmoscope. Asterisks, indicates the location of the optic disk, while open arrowheads, □ point toward the optic disc (not depicted in the micrograph). FIG. 10A depicts representative micrographs showing (a) a small number of unconjugated microparticles and (b) a comparably small number of PSGL-1-conjugated microparticles in the choriocapillaris of normal control rats. In the temporal area, the number of PSGL-1-conjugated microparticles accumulated in the choriocapillaris of EIU rats peaked at 4 h after LPS injection (c) and decreased gradually by 36 h after LPS injection (d). In contrast, in the central area, the number of PSGL-1-conjugated microparticles revealed a biphasic pattern with two peaks at 4 h (e) and 36 h (f) after LPS injection, respectively.

To determine whether it is possible to reveal the level of endothelial injury in the ocular vessels with the PSGL-1-conjugated microparticles, EIU was induced in rats and the number of adhering microparticles was quantified at different time points (FIG. 10A). In normal control animals, a low number of microparticles constitutively adhered to the endothelium of the choriocapillaris (14±1). In contrast, in the EIU animals, a large number of microparticles (132.8±9.5) accumulated in the temporal area (temporal mid-periphery) with a peak at 4 h after LPS injection, showing a significant 9.5-fold increase compared to the untreated control group (p=2.1×10⁻⁷) (FIG. 10B). FIG. 10B depicts temporal choriocapillaris microcirculation, showing average numbers of accumulated plain and PSGL-1-conjugated microparticles in healthy normal (0 h) and EIU animals at different time points (4, 10, 24, 36, and 48 h after LPS injection) in the temporal area of the choriocapillaris microcirculation of live rats. Values are mean±SEM, n=6 animals in each group, ‡ p<0.01.

In comparison, the number of accumulated microparticles in the central area (around the optic disk) peaked at 4 and 36 h after LPS injection (107.3±15.2 at 4 h, p=6.4×10⁻⁸, and 84.8±3.2, p=2×10⁻⁹ at 36 h), an increase of 6.9- and 5.5-fold compared to the control group, respectively (FIG. 10C). FIG. 10C depicts central choriocapillaris microcirculation, showing average numbers of accumulated plain and PSGL-1-conjugated microparticles in healthy normal (0 h) and EIU animals at different time points (4, 10, 24, 36, and 48 h after LPS injection) in the central area of the choriocapillaris microcirculation of live rats. Values are mean±SEM, n=6 animals in each group, ‡ p<0.01.

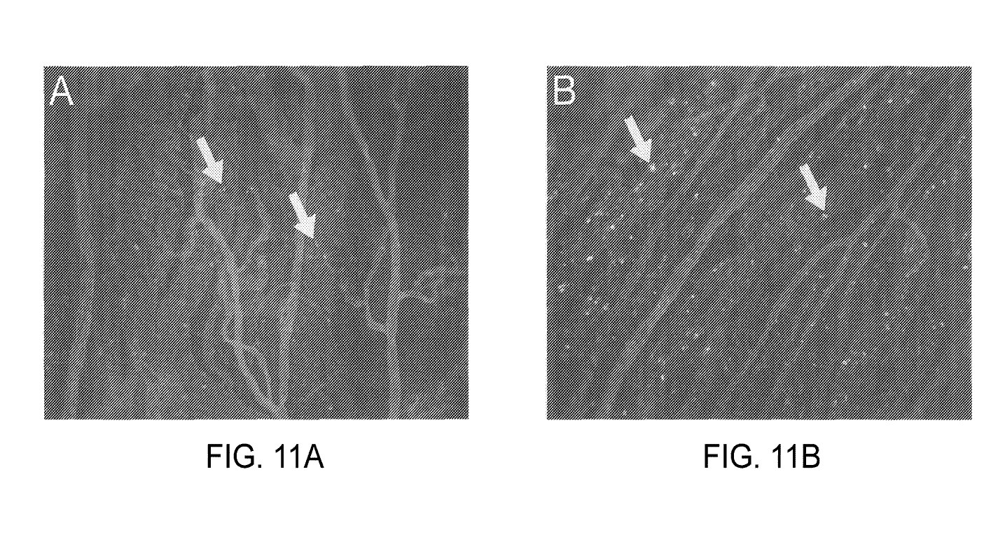

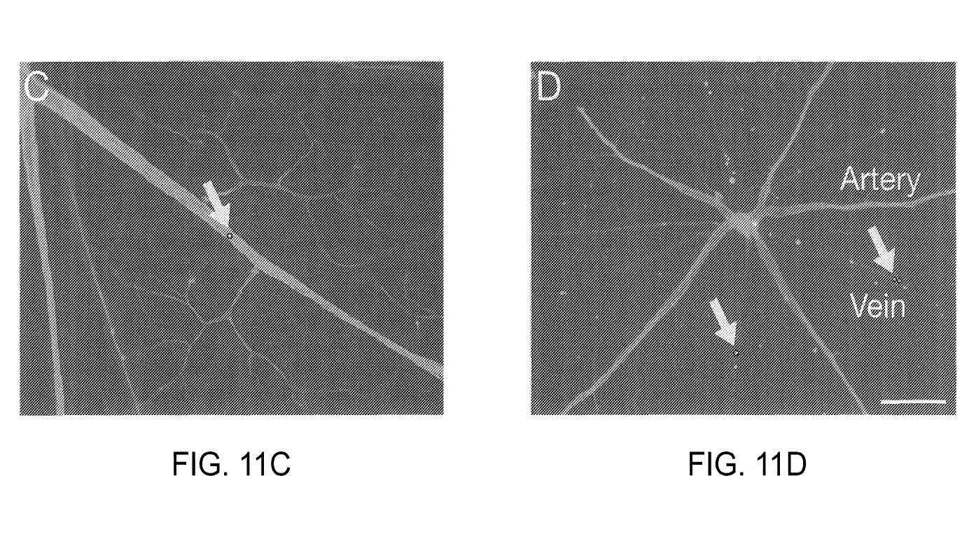

To confirm that the PSGL-1-conjugated microparticles, detected in vivo, were indeed in the choriocapillaris, as was postulated based on the depth of the SLO focus, the accumulation of the conjugated microparticles was further examined using the retinal and choroidal flatmount technique. An amount of 6×10⁸ PSGL-1-conjugated microparticles was injected through a tail vein catheter into EIU rats. Thirty minutes later, the animals were perfused with Rhodamine-coupled Con A to remove non-firmly adhering microparticles and to stain the endothelial surface. The animals' eyes were then enucleated and retinal and choroidal flatmounts were prepared. Using epifluorescence microscopy, it was possible to confirm the specific adhesion of microparticles in the retinal vessels and choriocapillaris (FIG. 11A-11D). FIGS. 11A-11D are micrographs depicting choroidal (A, B) and retinal (C, D) flatmounts of normal and EIU animals, respectively (4 h after LPS treatment) that were injected with microparticlcs (yellow arrows) through the tail vein. Animals were perfused with rhodamine-labeled Con A to stain the vasculature. FIGS. 11A and 11B show firmly adhering microparticles in the choriocapillaris of a normal animal (A), and an EIU animal (B), 4 h after LPS treatment. FIGS. 11C and 11D show firmly adhering microparticles in retinal vessels of a normal animal (C) and an EIU animal (D), 4 h after LPS treatment. The bar in FIG. 11D represents 100 μm.

In line with the SLO-findings, the flatmounts showed a large number of PSGL-1-conjugated microparticles accumulated in the retinal vessels and choriocapillaris of the EIU animals. Interestingly, the retinal flat-mounts revealed that nearly all firmly adhering microparticles had accumulated in the major retinal veins (FIG. 11C, D). This is consistent with the finding that, during EIU, most leukocytes are found in the retinal veins and suggests that the microparticles mimic the pathophysiologically relevant phenomenon of leukocyte recruitment in EIU.

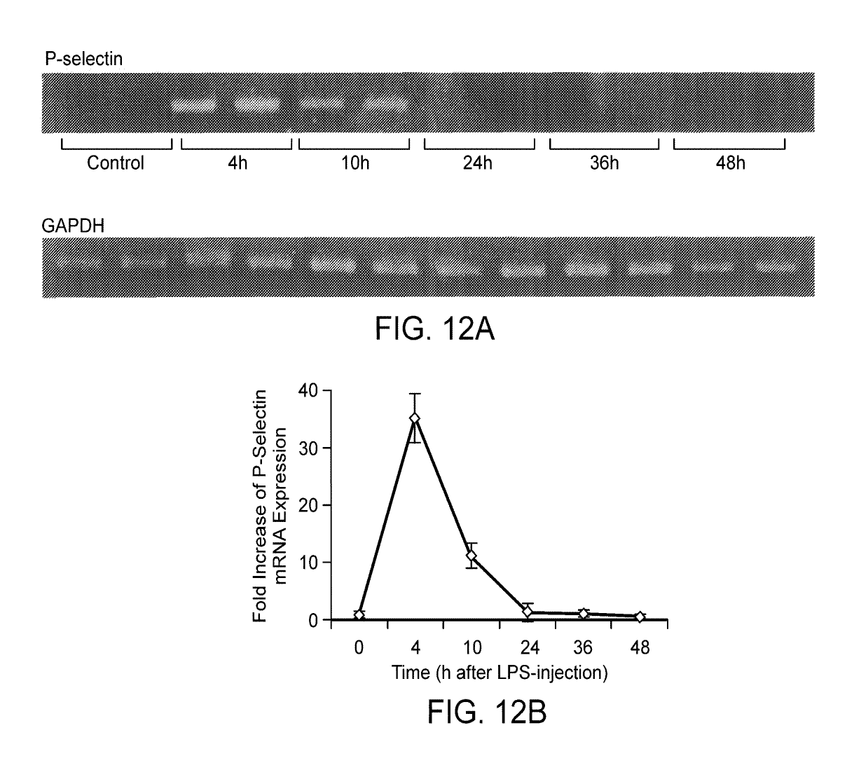

To investigate whether the changes in PSGL-1-conjugated microparticle recruitment during EIU reflect specific changes in endothelial antigen expression, the expression of P-selectin mRNA was semiquantified in the choroidal vessels of the EIU animals at various time points after LPS-injection, using PCR and gel electrophoresis (FIG. 12A-12B). Bands in FIG. 12A indicate the expression level of P-selectin and GAPDH mRNA in the choroidal tissues of rats at the indicated time points after LPS injection (control, no LPS-treatment). GAPDH was used as a control. FIG. 12B shows an increase of choroidal P-selectin mRNA-expression in LPS-treated animals compared to the non-LPS-treated controls (0 h) as determined by band densitometry. Data represent mean±SEM, n=3.

A peak expression of P-selectin mRNA was detected 4 h after LPS injection, corresponding to the high levels of microparticle accumulation in the choriocapillaris at this time point. These results suggest that the rolling and adhesion of the PSGL-1-conjugated microparticles in the choriocapillaris correlate with the endothelial P-selectin expression and is an indirect means for its quantification.

These experiments show that the early rise in the accumulation of PSGL-1 conjugated microparticles in EIU animals correlates with the endothelial P-selectin mRNA expression. The quantification of the microparticle-endothelial interactions in the choriocapillaris allows the estimation of the expression level of endothelial P-selectin, an established marker of vascular injury in living animals. However, since PSGL-1 also binds to E-selectin, it is possible that changes in microparticle accumulation may in part also reflect the level of expression of the endothelial E-selectin. Furthermore, PSGL-1 conjugated microparticles may accumulate in EIU animals by binding to L-selectin on the surface of firmly adhering leukocytes. Although at the current SLO resolution it may not always be possible to distinguish between interaction of the microparticles with endothelium vs. firmly adhering leukocytes, improvements of the imaging technique may allow such distinctions.

Interestingly, the measurements of the accumulation of the PSGL-1-conjugated microparticles revealed distinct patterns in different areas of the choriocapillaris flow. In the central area around the optic disk, microparticle accumulation peaked at 4 and 36 h after LPS injection, while the number of accumulated microparticles in the temporal area showed only one peak at 4 h after LPS injection, suggesting regionally diverse inflammatory responses within the choriocapillaris. These data indicate that the technique provides the sensitivity to detect subtle regional differences in inflammatory response at a functional level.

The experiments also show a higher level of microparticle-endothelial interactions in the choriocapillaris flow compared to that of retinal microcirculation during EIU. It is possible that the higher vascular density in the choriocapillaris may account in part for the higher number of accumulated microparticles. However, the drastic differences found suggest functional differences between these two distinct vascular beds.

An early peak in the expression of P-selectin mRNA is shown in the choroid of EIU animals. This is the first report of the measurement of P-selectin mRNA expression in the choroid during EIU. The mRNA results may not illuminate the subtle regional differences that were depicted in the in vivo experiments, as mRNA was collected from the entire choroidal complex. Furthermore, P-selectin is both de novo synthesized and rapidly released from cytosolic granules (Weibel-Palade bodies) upon endothelial activation. Immunohistochemistry shows P-selectin upregulation in the iris-ciliary body as early as 15 min and 5-7 h after LPS injection. While the first peak after 15 min is likely due to rapid release of P-selectin protein from the cytosolic granules, the second peak is consistent with the time course of mRNA upregulation in the experiments.

Using the method described herein, the rolling flux of PSGL-1-conjugated fluorescent microparticles was quantitatively evaluated in the choriocapillaris flow and its peak time was determined to be at 4-10 h after LPS injection. Interestingly, previous studies with acridine orange digital fluorography showed that the number of rolling leukocytes in the retina of LPS-treated rats peaks 12 h after LPS injection. Since the results herein indicate an earlier peak than the acridine orange-labeled leukocytes in the retinal vessels, the present technique may thus allow an earlier detection of endothelial changes than conventional visualization techniques of leukocyte-endothelial interaction in vivo.

Consistent with the previously reported lobelike structure of the choriocapillaris, in the above experiments, all rolling microparticles moved within confined areas of the choriocapillaris, which may correspond to the area of the anatomical lobe.

The technique described herein allows convenient and quantitative imaging of adhesion molecule expression on the endothelium of the choriocapillaris in vivo. The fact that a variety of adhesion molecules or antibodies can be conjugated to the microparticles makes this technique a versatile and powerful tool for the study of the expression and function of endothelial surface antigens and detection of endothelial injury during disease.

A minimally invasive technique can be used to image the early stages of endothelial dysfunction during DR or other ocular inflammatory diseases by targeting antigens on the injured retinal and choroidal endothelium and accumulated leukocytes. ICAM-1 and VCAM-1 expression on retinal vessels are elevated in response to experimentally-induced diabetes and may predict progression of disease. The currently available experimental approaches to detect over-expression of these and other endothelial markers are not applicable to the clinical setting. A minimally invasive means to detect the increased expression of endothelial markers allows monitoring of early indicators of endothelial dysfunction during DR or other inflammatory diseases. Furthermore, targeting known leukocyte markers such as CD18 and VLA-4 allows detection and quantification of firmly adhering leukocytes during DR. Additionally, should specific endothelial markers of ocular diseases become available, the technique allows their targeting as well. These approaches allow an earlier clinical staging of DR than currently possible.

Further experiments can be conducted to develop further methods for sub-clinical diagnosis of DR. For example, diabetes can be induced in Long Evans rats and the rolling and adhesion parameters of fluorescent microparticles in their retinal and choroidal vessels can be examined at various durations of disease. To detect specific retinal and choroidal endothelial antigens, such as ICAM-1, VCAM-1 and P-selectin in normal and diabetic animals, microparticles can be conjugated with commercially available ligands of the endothelial antigens, such as monoclonal antibodies or recombinant CD18, VLA-4 and PSGL-1. To visualize the microparticles interacting with endothelial antigens, they can be injected into live anesthetized animals and images obtained of the fundus of these animals using SLO. In addition, retinal and choroidal flatmounts of perfused diabetic and normal animals that were injected with these microparticles can be made and the number of microparticle and leukocyte adhesions quantified to validate the results obtained with SLO. As a positive control, similar in vivo microparticle tracking by SLO and flatmount experiments can be performed after injections of proinflammatory mediators, such as TNF-α, into the vitreal cavity or LPS into the footpad to induce retinal inflammation and the expression of endothelial adhesion molecules. Another control can be used to assess the specificity of microparticle binding to the inflamed endothelium. To do so, the endothelial antigens can be blocked (e.g. with mAbs) or animals deficient for these molecules can be used.

Functional microparticle data can be correlated with expression of specific endothelial molecules by isolating retinal and choroidal tissues from the experimental animals and semi-quantifying mRNA or protein of the targeted endothelial molecules, as detailed below.

Fluorescent microparticles can be used to detect firmly adhering leukocytes in the retinal and choroidal vessels during diabetes. To further develop methods of detecting and quantifying retinal and choroidal leukocyte accumulation as an indicator of the level of vascular injury during the early stages of diabetes, the number of firmly adhering fluorescent microparticles to specific leukocyte antigens, such as CD18 and VLA-4, can be quantified in normal and diabetic animals. To detect these leukocyte antigens in vivo, the microparticles can be coated with commercially available ligands of CD18, VLA-4, and PSGL-L. To visualize the microparticle interaction with firmly adhering leukocytes, live images of the fundus of normal and diabetic rats can be obtained using SLO, and flatmounts can be prepared from the retinas and choroids of these animals to determine what percentage of the microparticles interact specifically with the leukocytes vs. non-specifically with the endothelium (see FIG. 12).

Thus, the quantitative evaluation of microparticle rolling and adhesion in the retinal and choroidal vessels of rats can be performed at different time points after diabetes induction. This evaluation provides direct visualization of the amount of endothelial injury in vivo. The following provides a more detailed description of various further experiments that can be conducted.

Specific endothelial antigens can be targeted to visualize retinal and choroidal vascular injury. Additional experiments can be performed in normal and diabetic animals to show the in vivo retinal and choroidal antigen expression during disease. The well-characterized streptozotocin-induced model of diabetes in rats can be used. To induce diabetes, Long-Evans rats (Charles River, Wilmington, Mass.), weighing 200-250 g can be fasted overnight and receive single intraperitoneal injections of streptozotocin (60 mg/kg; Sigma, St. Louis, Mo.) in 10 mM citrate buffer (pH 4.5). Control non-diabetic animals can receive citrate buffer alone. To confirm the diabetic state, the blood glucose level can be measured before each experiment, and only animals with levels of 250 mg/dL or higher after streptozotocin injections would be considered diabetic and included in the study. Two weeks after diabetes induction, animals can be anesthetized with Xylazine hydrochloride (4 mg/kg) and Ketamine hydrochloride (10 mg/kg), and their pupils can be dilated with 0.5% Tropicamide and 2.5% Phenylephrine hydrochloride. Each animal can have a catheter inserted into the tail vein through which 10⁸ microparticles conjugated to ICAM-1 specific mAb or other types of previously suggested molecules can be injected. The fundus of these animals can then be imaged by SLO at 488 nm and 30° field of view angle. A contact lens can be used to retain corneal clarity throughout the experiment. Based on a power calculation, 8-10 rats per group (i.e. diabetic vs. normal) are estimated to be necessary to achieve meaningful results per targeted antigen. These estimates take into account a rate of failure of about 20% due to non-responsive blood-glucose levels, death during anesthesia or other less frequent complications. For these sets of experiments at least 3 known endothelial antigens associated with ocular inflammation can be targeted—namely ICAM-1, VCAM-1, and P-selectin—or other novel retinal DR-specific antigens. These sets of experiments would require, for example, approximately 60-80 Long Evans rats. Retinal and choroidal vessels can be evaluated in the same animals.

Specific leukocyte antigens can be targeted to visualize retinal and choroidal leukocyte accumulation. For example, to assess whether fluorescent microparticles can reveal adhering leukocytes during diabetic retinopathy, diabetes can be induced in Long Evans rats with the streptozotocin technique, as detailed above, and the interaction of the microparticles with leukocyte antigens that are numerically or functionally upregulated during diabetes, such as CD18 and VLA-4, can be examined. To do so, the microparticles can be coated with commercially available ligands of CD18 and VLA-4, such as mAbs (Seikagaku America, Cape Cod; Pharmingen, USA) or recombinant ICAM-1 or VCAM-1 (R&D Systems, USA). Live images of the fundus of normal and diabetic animals can be obtained using an HR2 SLO device. In addition, flatmounts of the retinas of these animals can be prepared, and the adhesion of the microparticles to leukocytes evaluated to validate the in vivo results. Similar experiments can be performed using intravitreal injections of proinflammatory mediators, such as TNF-α, to induce inflammation and leukocyte accumulation, as a positive control, described in more detail below. These sets of experiments would require approximately 50-60 Long Evans rats.

Microparticles conjugated to antibodies or ligands of CD18 or VLA-4 interact with leukocytes that are firmly adhering to the endothelium of diabetic retinal vessels (see FIG. 12).

To test the specificity of the microparticle interactions, it is possible to block the antigens with neutralizing mAbs or inject the conjugated microparticles into animals deficient for the ligands of the conjugated proteins. In order to be able to use mice deficient for ICAM-1, P-selectin, and CD18 (Jackson Laboratories, Bar Harbor, Me.), the visualization technique can be adapted for murine use. For example, the use of SLO to visualize retinal vessels in mice requires an optical adjustment due to the higher refraction of the murine cornea. To correct for the higher refraction, commercial mouse contact lenses can be used (e.g., BC 1.65 mm, size 3.0 mm, Power +20.0; Unicon Corporation, Osaka, Japan). Furthermore, induction of diabetes in mice is different from that in rats; for instance it requires several Streptozotocin injections to prevent recovery of the animals from the diabetic state as opposed to the single injection for rats. Using this protocol, diabetes can be stably generated in mice, as confirmed by regular blood glucose measurements.

For faster and more convenient optimization of the experimental conditions, such as determining the optimal coating-density and microparticle numbers, prior to their application in the more elaborate diabetic model, experiments can be conducted in a well-established acute model of ocular inflammation, the Endotoxin Induced Uveitis (EIU). EIU experiments also provide a model of inflammation to compare with the visualization experiments in early DR. To induce EIU, 100 μl (2 mg/ml) lipopolysacharide (LPS) can be injected into the footpad of Lewis rats. Control animals can receive equal volumes of saline. The retinal inflammation as quantified by leukostasis peaks at 24 h after LPS injection, the time point at which the visualization experiments can be performed.

To visualize firmly adhering leukocytes and interacting microparticles in a side-by-side manner, retinal and choroidal flatmounts can be prepared from diabetic and EIU animals after the microparticle experiments with SLO. Due to the high resolution that can be achieved under light microscopy with these flatmounts, it is possible to directly visualize the binding of microparticles to adherent leukocytes or endothelium (see FIGS. 9 and 12).

Perfusion of the animals, staining of the vessels and leukocytes, and preparation of retinal and choroidal flatmounts can be performed. Images of the retinal microvessels can be obtained using epifluorescence microscopy, and the total number of adherent leukocytes per retina can be determined, as shown in FIG. 12.

To correlate the functional microparticle interaction data with the expression of endothelial molecules, semiquantification of endothelial genes, such as P-Selectin or ICAM-1, can be performed. For example, one eye from each animal can be enucleated and total RNA can be isolated from the retina or choroid. Each first-strand reaction can be amplified using P-selectin-, ICAM-1, VCAM-1 and GAPDH-specific oligonucleotide primers. The reactions can be analyzed by agarose gel electrophoresis and ethidium bromide staining to determine the levels of transcript relative to the control. Protein levels can be determined using commercially available ELISAs.

Experiments can be performed to detect progression of disease during the early stages of DR. Diabetic rats tend to lose a significant amount of weight and dehydrate due to the increased urine production. For experiments requiring longer periods of diabetes than 2 weeks, it is important to be able to maintain the animals in a stable condition. This can be achieved, for example, by regular insulin administration.

The amount of endothelial injury can be detected by microparticle interaction and can be quantified as the flux, rolling velocity, and the number of firmly adhering microparticles.

Microparticles conjugated to antibodies or ligands of endothelial antigens interact with the endothelium of diabetic retinal and choroidal vessels. The quality and the quantity of the interactions are dependent on the extent of the endothelial injury. This interaction is specific and will likely increase with the length of the period after diabetic induction. Early stages of disease may thus be found to correspond with lower numbers of rolling and firmly adhering microparticles, whereas later stages of disease may correspond with larger numbers of interactions.

In certain embodiments, methods of the invention require the use of fluorescent microparticles in vivo, for example, in combination with an SLO device. For example, experiments have been conducted using carboxylated monodispersed polystyrene fluorescent microparticles (Fluoresbrite®; Polysciences, Warrington, Pa.) are available in different sizes and with a variety of fluorescent properties. Other fluorescent microparticles may be used, for example, those that are currently approved for clinical use, such as FDA approved albumin-shelled microbubbles, currently used in cardiac imaging.

The microparticles can be conjugated with binding partners of interest, for example, the binding partners described herein, using coupling chemistries known to those skilled in the art.

In regard to the size of rigid microparticles, since they lack the visco-elasticity of leukocytes, they are not able to deform to fit through capillaries with a smaller diameter than their own. Therefore, rigid microparticlcs of similar dimensions as leukocytes would block capillaries and cause non-perfusion. To prevent this, rigid microparticles that are significantly smaller in diameter than capillary diameters should be used. For example, microparticles that have average diameter (e.g., number average) of about 10 μm or less, about 7 μm or less, about 5 μm or less, about 4 μm or less, about 3 μm or less, about 2 μm or less, or about 1 μm or less, may be used. Alternately, elastic (e.g., flexible and/or viscoelastic) microparticlcs may be used, with or without the above limitation on diameter as long as they are small enough to pass through the capillaries (e.g., they have diameter less than the capillary diameter). For example, in various embodiments, elastic microparticles that have average diameter of less than or equal to about 50 μm, less than or equal to about 40 μm, less than or equal to about 30 μm, less than or equal to about 20 μm, or less than or equal to about 10 μm may be used.

Additionally, an adequate signal strength for detection is necessary; therefore, a lower limit of average microparticle diameter may be applicable (e.g., no less than about 0.01 μm, no less than about 0.05 μm, no less than about 0.1 μm, no less than about 0.5 μm, no less than about 1 μm, no less than about 2 μm, or no less than about 3 μm). However, in certain embodiments, microparticles having diameter less than 0.01 μm may be used. Microparticles that are usable may include, for example, those with average diameter from 0.5 μm to 5 μm, from 1 μm to 5 μm, from 2 μm to 5 μm, from 3 μm to 5 μm, from 0.5 μm to 3 μm, from 1 μm to 3 μm, from 2 μm to 3 μm. Signal may also be dependent on the conjugation of binding partners on the surface of the microparticles, and the loading can be adjusted accordingly. The microparticles need not necessarily be spherical in shape. For example, microparticles may have a flattened or semi-flattened surface, and/or they may be irregular in shape. The microparticles may be hollow, partially hollow, or filled (solid), for example. The microparticles may be solid shells with gas interiors. The microparticles may be filled with one or more compounds to be delivered to the addressed vascular areas. In alternate embodiments, the microparticles may be liquid.

In certain embodiments, the microparticles used can be, or can have features of, the microparticles currently used in echocardiography applications, for example, those originally described in R. Gramiak, P. M. Shah, “Echocardiography of the aortic root,” Invest. Radiol., 3, 356-366, (1968), the text of which is incorporate herein by reference in its entirety. Modifications and changes in material properties have been made, including a higher stability and a more effective delegability of these agents via ultrasound methods.

For example, the microparticles may constitute microparticles of about 1 to about 4 microns in diameter, which axe enclosed by a lipid, polymer or protein shell.

These microparticles, often also referred to as microbubbles, can be filled with a variety of gases, which may provide one or more acoustic scattering signatures, helping to distinguish them from the acoustic properties of plasma, blood cells or the surrounding tissues. Alternately, unencapsulated gas bubbles may be used. Rapid dissolution of these shell-free microbubbles after their systemic injection may limit their applicability for some uses. To provide stability and increase the in vivo half-life of the microparticles, various biocompatible materials can be introduced as a protective outer layer. Furthermore, the microparticles may be made to contain heavy molecular weight gases such hexafluorides (S. Mayer, P. A. Graybum, “Mycocardial contrast agents: recent advances and future directions,” Prog. Cardiovascular Dis., 44, 33-44, 2001., the text of which is incorporated herein by reference in its entirety for all purposes). Such hardshell microparticles with a gas interior have resonance frequency in the MHz range (e.g., this may be important where they are detected by ultrasound techniques). Furthermore, they have unique physical properties, including a non-linear oscillation of their size around their equilibrium radii, a detectable second or higher harmonic wave, and also subharmonic waves in response to ultrasound.

Most contrast agents currently used in cardiac imaging range between 1-3 μm in diameter and resonate in frequencies in the range 1-5 MHz.

An example SLO that can be used in various embodiments described herein is the Heidelberg Retina Angiograph 2 (HRA2; Heidelberg Engineering, Germany). The HRA2 is a confocal laser-scanning device that emits laser light of three different wavelengths (458, 795, and 830 nm). For certain of the experiments presented herein, the blue line of the solid-state laser was used at 488 nm to excite microparticles with a maximum excitation wavelength of 441 nm. A barrier filter at 500 nm edge wavelength was used to separate excitation from the fluorescent light of microparticles to achieve an enhanced signal to noise ratio. The microparticles chosen for the experiments are clearly visible. However, microparticles with an excitation wavelength more closely matching that of the blue laser can be used. This may generate a stronger emission from the microparticles and ultimately allow use of smaller microparticles, if desired.

The HRA2 SLO device allows a maximum resolution of 1536² pixels, with a pixel being the equivalent of 5.7 μm of the retinal surface, independent of the field of view angle (15-30°). Furthermore, the HRA2 allows up to 16 frames/sec at a resolution of 3682 pixels and a field of view of 15°. From experiments described herein, a frame rate of 10/sec or higher is sufficient to distinguish interacting microparticles (i.e. firmly adhering or rolling) from freely flowing ones and to obtain rolling velocities from those interacting. Therefore, to distinguish rolling microparticles in the SLO, the High Speed Mode can be used at a 20-30° field of view angle.

Carboxylate groups on the surface of the microparticles can be used to covalently couple them to Protein G (Sigma, P-4689), using a carbodiimide coupling kit (Polysciences, #19539). The various antibodies or Fc-coupled recombinant molecules (i.e. R&D Systems, Minneapolis, Minn. and Y's Therapeutics, Co. Ltd., Tokyo, Japan) can be coated onto the microparticles by incubating microparticles with Fc-coupled constructs (at 0.1 mg/ml) in PBS for 15 min at 37° C. Microparticles can be used after wash in PBS with 10% FBS.

After the conjugation process, a flow cytometer (B&D) can be used to quantify the number of bound peptides, proteins, or antibodies on the surface of the microparticles. The flow cytometer can be calibrated using the Quantum Simply Cellular kit (FCSC #815, Fischers, Ind.) in combination with the provided software (QuickCal) for regression analysis.

The preliminary experiments suggest that the systemic administration of 10⁸ microparticles is sufficient for in vivo imaging. However, it is possible that lower numbers of microparticles may suffice to provide a high enough accumulation of microparticles in the retina to allow statistical analysis, with a minimum amount of excess microparticles in the circulation.

To further reduce the background interaction of microparticles under control conditions, microparticles with less interactive surface moieties can be used.

Optionally, intravitreal injections of a proinflammatory cytokine, TNF-α (500 ng in 5 μl saline), can be used to induce retinal inflammation in one rat eye, while the contra lateral eye remains untreated. These experiments would reduce inter-individual variability, since the treated and untreated eyes are in the same animal. Varying numbers of conjugated microparticles can be administered systemically to the rats and their binding to the retinal vessels in both eyes can be quantified by SLO. To perform intravitreal injections a 33-gauge double-caliber needle (Ito Corp., Fuji, Japan) can be inserted into the vitreous approximately 1 mm posterior to the corneal limbus. Insertion and infusion can be directly viewed under an operating microscope (Leica, Germany).

Software for automated particle tracking can be used to analyze SLO recorded images. Interacting microparticles are easily discernible by their characteristic gradual displacement in subsequent frames and due to their significantly lower rolling velocity compared to the midstream free-flowing microparticles. The number of rolling microparticles will be counted for 30 s. Rolling microparticles will be followed for several frames by freeze frame advancing to calculate microparticle rolling velocities, defined as the traveled distance divided by the tracking time (FIG. 12). Rolling velocities of 25 or 50 microparticles can be measured in various vessels (10-50 μm diameter), sorted and averaged for each rank to construct cumulative histograms. The number of firm adhesions can be counted under various experimental conditions in different areas of the same fundus (temporal and central regions) and can be averaged, for example, 30 minutes after microparticle injection. The interaction flux, defined as the number of interacting microparticles per time, can be measured in each experiment.

To ensure similar hemodynamic conditions between the controls and experimental groups, the maximal blood flow velocity can be measured in the vessels of interest by freeze-frame tracking of freely flowing microparticles in the center of the vessel. Measurements can be used to compute volume flow rate and shear forces.

The prevailing shear forces in certain vessels, especially in larger veins and arteries, may be too high to allow binding of certain microparticles to some endothelial ligands. This may prevent the visualization of molecules despite their presence on the endothelium. In such a scenario, smaller microparticles may be used, which would be less affected by the shear forces, and thus may allow visualization of the antigens. Alternatively, the site density of the conjugated molecules on the microparticles can be increased, which would lead to formation of more bonds between the microparticle and the endothelium.

Comparisons of groups can be performed using known statistical techniques, for example, paired or unpaired Student's t-test, where appropriate.

In certain embodiments, the imaged endothelial surface antigens can be used to assess the effectiveness of therapeutic interventions. Animal studies may include providing some rats with a therapeutic insulin regimen that is known to prevent retinal abnormalities. The panel of endothelial surface antigens may be imaged in these animals and compared with untreated diabetic controls.

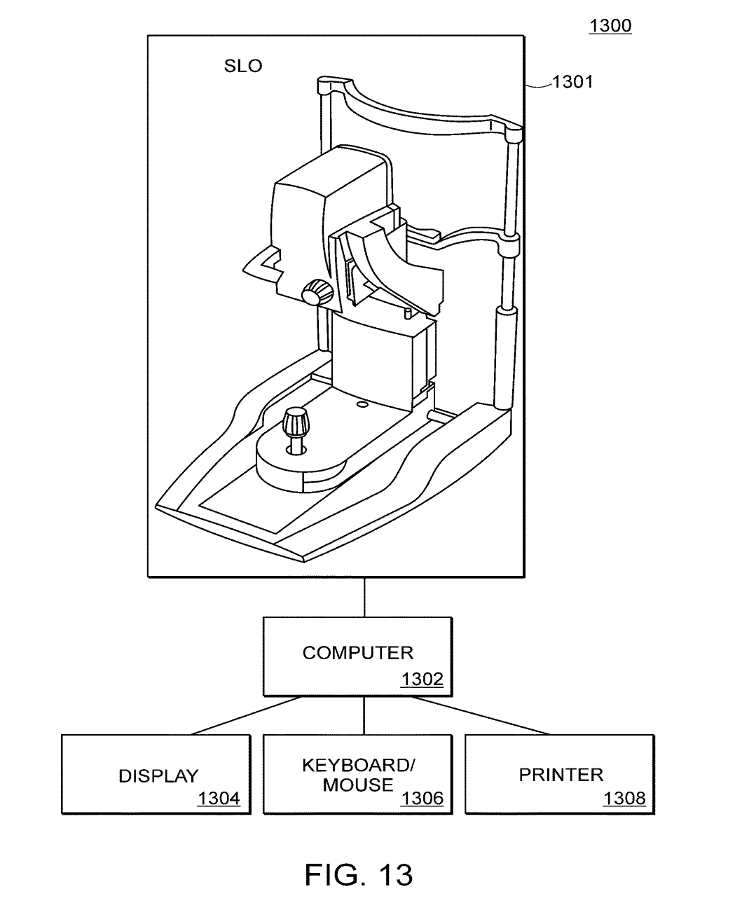

FIG. 13 is a block diagram of a scanning laser ophthalmoscope (SLO) system 1300 for use in certain embodiments of the methods described herein. The SLO 1301 may be, for example, the Heidelberg Retina Angiograph 2 (HRA2; Heidelberg Engineering, Germany). The SLO system 1300 also includes a computer 1302 which executes software that may control operation of the system and/or analysis of results. The software includes one or more modules recorded on machine-readable media such as magnetic disks, magnetic tape, CD-ROM, and semiconductor memory, for example. Preferably, the machine-readable medium is resident within the computer. In alternative embodiments, the machine-readable medium can be connected to the computer by a communication link (e.g., via the internet). In alternative embodiments, one can substitute computer instructions in the form of hardwired logic for software, or one can substitute firmware (i.e., computer instructions recorded on devices such as PROMs, EPROMS or EEPROMs, or the like) for software. The term machine-readable instructions as used herein is intended to encompass software, hardwired logic, firmware and the like.

The computer 1302 in FIG. 13 may be a general purpose computer. The computer can be an embedded computer, a personal computer such as a laptop or desktop computer, of other type of computer, that is capable of running the software, issuing suitable control commands, and recording information in real time. In one embodiment, the computer has a display 1304 for reporting information to an operator of the SLO system, a keyboard 1306 for enabling the operator to enter information and commands, and/or a printer 1308 for providing a print-out, or permanent record, of measurements made by the SLO system and for printing micrographs, images, or results, for example.

In certain embodiments, the invention is directed to a method of targeted substance (e.g., drug) delivery to a portion of an intraluminal surface of a blood vessel (e.g., an injured portion). The method involves administering to a subject microparticles carrying one or more drugs or other agents, where the microparticles have a surface to which one or more binding substances are conjugated. The one or more binding substances bind to one or more ligands on a targeted intraluminal surface of the blood vessel as described in more detail herein above, thereby immobilizing the microparticles on the targeted intraluminal surface. Once the microparticles are immobilized, the release of the one or more agents from the microparticles onto the targeted intraluminal surface may be affected, for example, by administration of laser light (or any other electromagnetic radiation), a magnetic field, and/or a releasing agent. The release may also be affected by passage of time, where the microparticles break down over time allowing diffusion of the agent held within the microparticles onto/into the targeted region. Although microparticles bind to the intraluminal surfaces, the released substance/drug can diffuse to the vicinity (e.g., the endothelium, vascular wall, and/or the tissue surrounding the blood vessels, and the targeted region for microparticle binding may therefore be different from the targeted region for substance/drug delivery.

For example, the substances carried by the microparticles can be radioisotopes for treatment of neoplasm (e.g., ocular melanoma of other solid cancer). The radioisotopes would not need to be physically released from the microparticles, but the radiation would be released to surrounding tissue over time. The presence of the radio-isotopes in the vicinity of the tumor would be therapeutically beneficial.

In certain embodiments, drugs or other substances are delivered to injured endothelium during acute or chronic inflammation, for example, uveitis, using markers of inflammation, such as selectins and their ligands, integrins and their ligands, etc. In other embodiments, drugs or other substances are delivered to injured endothelium during diabetic retinopathy or AMD using markers of neovascularizations, such as the αᵥβ₃ integrin.

Drugs or other substances that may be delivered to targeted regions include, for example, autonomic drugs; cardiovascular-renal drugs; drugs affecting inflammation; drugs that act in the central nervous system; drugs that treat diseases of the blood, inflammation, and gout; drugs acting on the blood and blood-forming organs; endocrine drugs; chemotherapeutic drugs; perinatal and pediatric drugs; geriatric drugs; dermatologic drugs; drugs used in the treatment of gastrointestinal diseases; and botanicals (herbal medications) and nutritional supplements including drugs of holistic medicine and homeopathy.

Autonomic drugs include, for example, cholinoceptor-activating & cholinesterase-inhibiting drugs, cholinoceptor-blocking drugs, adrenoceptor-activating & other sympathomimetic drugs, adrenoceptor antagonist drugs, general anesthetics, local anesthetics, therapeutic gases (oxygen, carbon dioxide, nitric oxide, and helium), agents to treat psychosis and mania, anti-depression and anxiety drugs, drugs in the treatment of central nervous system degenerative disorders.

Cardiovascular-renal drugs include, for example, antihypertensive agents, vasodilators & agents for the treatment of angina pectoris, drugs used in heart failure, agents to treat congestive heart failure, agents used in cardiac arrhythmias, diuretic agents, drugs impacting smooth muscle action, histamine, serotonin, & the ergot alkaloids, vasoactive peptides, eicosanoids (prostaglandins, thromboxanes, leukotrienes, & related compounds, nitric oxide, drug therapy for hypercholesterolemia and dyslipidemia.

Drugs affecting inflammation include, for example, drugs used in asthma, histamine and histamine receptor agonist and antagonists, bradykinin, and their antagonists, lipid-derived autacoids (eicosanoids and platelet-activating factor), analgesic-antipyretic and antiinflammatory agents; pharmacotherapy of gout.

Drugs that act in the central nervous system include, for example, sedative-Hypnotic Drugs, alcohols, antiseizure drugs, general anesthetics, local anesthetics, skeletal muscle relaxants, drugs for the management of parkinsonism & other involuntary or voluntary movement disorders, antipsychotic agents & lithium, antidepressant agents, opioid analgesics & antagonists, drugs for treatment of addictions.

Drugs used to treat diseases of the blood, inflammation and gout include, for example, agents used in anemias; hematopoietic growth factors, drugs used in disorders of coagulation, agents used in hyperlipidemia, nonsteroidal anti-inflammatory drugs, disease-modifying antirheumatic drugs, and nonopioid analgesics.

Drugs acting on the blood or blood-forming organs include, for example, hematopoietic agents, growth factors, minerals, and vitamins, blood coagulation and anticoagulant, thrombolytic, and antiplatelet drugs.

Endocrine drugs include, for example, hypothalamic & pituitary hormones and their hypothalamic releasing hormones, thyroid & antithyroid drugs, adrenocorticosteroids & adrenocortical antagonists, gonadal hormones & inhibitors, pancreatic hormones & antidiabetic drugs, agents that affect bone mineral homeostasis, estrogens and progestins, androgens, adrenocorticotropic hormone; adrenocortical steroids and their synthetic analogs; inhibitors of the synthesis and actions of adrenocortical hormones, insulin, oral hypoglycemic agents, and agents affecting the endocrine or exocrine pancreas function, agents affecting mineral ion homeostasis and bone turnover.

Chemotherapeutic drugs include, for example, penicillins, cephalosporins and other—lactam antibiotics & other cell wall—& membrane-active antibiotics, tetracyclines, inhibitors of protein synthesis, macrolides, clindamycin, chloramphenicol, & Streptogramins, aminoglycosides & spectinomycin, sulfonamides, Trimethoprim, & Quinolones, antimycobacterial drugs, antifungal agents, antiviral agents (nonretroviral, and antiretroviral agents in the treatment of HIV), other antimicrobial agents, antiparasitic drugs, antiprotozoal drugs (i.e. drugs against malaria, amebiasis, giardiasis, trichomoniasis, trypanosomiasis, leishmaniasis, and other protozoal infections), anthelmintic drugs, drugs against tuberculosis, mycobacterium avium complex disease, and leprosy, chemotherapy of neoplastic diseases, and cancer chcmotherapeutic drugs, immunopharmacology and Immunomodulatory drugs, immunosuppressants, tolerogens, and immunostimulants.

Drugs used in the treatment of gastrointestinal diseases include, for example, pharmacotherapy of gastric acidity, peptic ulcers, and gastroesophageal reflux disease, treatment of disorders of bowel motility and water flux; antiemetics; agents used in biliary and pancreatic disease, pharmacotherapy of inflammatory bowel disease.

Ligands on the intraluminal surface that can be targeted for the molecular imaging and/or targeted substance delivery methods described herein include those that are known to be associated with a particular disease of interest, as well as those that will become known to be associated with a disease of interest. Example ligands include angiogenesis related molecules and their receptors, for example, 4N1K, AGF (angiopoietin-related growth factor), Angiogenin (ANG), Angiopoietin-1, Angiopoietin-2, Angiostatin, ARP4 (angiopoietin-related protein 4), bFGF (basic fibroblast growth factor), CD31 (PECAM-1), CD34, CD97, CD146 (MUC18), Collagenase-1 (C1), COX-2 (Cyclooxygenase-2), Extra-Domain B (ED-B) of Fibronectin, Endoglin (CD105), ESAF (Endothelial cell stimulating angiogenesis factor), Factor VIII, Fit-1 (Fins-like tyrosine kinase 1), Integrin alpha1, alpha2. Integrin alpha2beta1, Integrin alpha3beta1, Integrin alpha 5 beta 1, Integrin alpha(v)beta(3), Integrin alpha6beta4, Integrin alpha9beta1, Integrin-beta(I), KDR, N-Cadherin, Ncstin, NG2 proteoglycan, PSMA (prostate-specific membrane antigen), PV-1 (Plasmalemmal vesicle associated protein-1), S100A13, Syndecan-1, T-Cadherin, TEM-5 (Tumor endothelial marker 5), TEM-8 (Tumour endothelial marker-8), Thrombospondin-1 (TSP1), Thrombospondin-2 (TSP2), Thy-1, Tie-1, Tie-2, Tn-C(Tenascin-C), TP (Thymidine phosphorylase), VCAM-1 (vascular cell adhesion molecule-1), VE-cadherin, VEGF, and VWF (von Willebrand Factor). Other examples of ligands on the intraluminal surface that can be targeted for the molecular imaging and/or targeted substance delivery methods described herein include cellular markers such as Adhesion/Extracellular Matrix-Associated Molecules (i.e. fibrinogen, fibronectin, galectins, integrins, junctional adhesion molecules, selectins, mucins, immunoglobulins), cytokine and chemokinc receptors, erythrocyte and other blood group antigens, apoptosis-associated molecules, epithelial cell-associated molecules, immunoglobulins, MHC antigens, T-Cell Receptor, leukocyte enzyme-associated molecules, leukocyte-associated molecules, megakaryocytc/platelet-associated molecules, multi-drug resistance-associated molecules, NK Cell-associated molecules, cytokines, cell proliferation markers, DNA, stem cell associated antigens, Alpha-2C-drenergic receptor (ADRA2C), ATP-binding cassette sub-family B (MDR/TAP) member 10 (ABCB10), ATP-binding cassette sub-family B (MDR/TAP) member 11 (ABCB11), ATP-binding cassette sub-family B (MDR/TAP) member 4 (ABCB4), ATP-binding cassette sub-family B (MDR/TAP) member 6 (ABCB6), ATP-binding cassette sub-family B (MDR/TAP) member 7 (ABCB7), ATP-binding cassette sub-family B (MDR/TAP) member 9 (ABCB9), ATP-binding cassette sub-family B (MDR/TAP) transporter 1 (TAP1), ATP-binding cassette sub-family B (MDR/TAP) transporter 2 (TAP2), ATPase alpha polypeptide Cu++ transporting (ATP7A), ATPase alpha polypeptide Cu++ transporting (ATP7B), ATPase class V type 10A (ATP10A), ATPase type 2C member 1 (ATP2C1), BK channel beta 1 subunit (Kenmb1), Calcitonin receptor (CALCR), CD151 antigen (CD151), CD28 antigen (CD28), CD34 antigen (CD34), CD72 antigen (CD72), EGF, latrophilin and seven transmembrane domain containing 1 (ELTD1), Endothelial differentiation sphingolipid G-protein-coupled receptor 1-8 (EDG1-8), Frizzled homolog 1 (FZD1), Frizzled homolog 7 (Fzd7), G protein coupled bile acid receptor 1 (GPBAR1), G protein-coupled receptor 4 (GPR4), G protein-coupled receptor 44 (GPR44, CRTH2), G protein-coupled receptor 73 (GPR73), G protein-coupled receptor 74 (GPR74), G protein-coupled receptor HM74a (HM74a), G-protein coupled purinergic receptor P2Y 8 (P2RY8), GPCR putative chemokinc receptor HM74 (HM74), GPCR putative chemokine receptor HM74 (HM74) polyclonal antibody, jagged 1 (JAG1), Jagged 2 (JAG2), Junction cell adhesion molecule 2 (Jcam2), Kangai 1 (KAI1, CD82), Leptin receptor (LEPR), low-density lipoprotein receptor-related protein 1 (LRP1), Low-density lipoprotein receptor-related protein 15 (LRP15), low-density lipoprotein receptor-related protein 2 (LRP2), low-density lipoprotein receptor-related protein 3 (LRP3), low-density lipoprotein receptor-related protein 4 (LRP4), low-density lipoprotein receptor-related protein 5 (LRP5) polyclonal antibody, Low-density lipoprotein receptor-related protein 6 (LRP6), Low-density lipoprotein receptor-related protein 8 (LRP8), Melanocortin 3 receptor (MC3R), Mouse mammary tumor virus receptor homolog 1 (MTVR1), patched homolog (PTCH) polyclonal antibody, patched homolog (PTCH), Patched homolog 2 (PTCH2), Phospho-integrin B4 [Y1492] (IGTB4), Phospho-integrin B4 [Y1510] (IGTB4), Phospho-integrin B4 [Y1596] (IGTB4), Phospho-integrin B4 [Y1712] (IGTB4), Phospho-tyrosine PDGFR [Y579] (PDGFRB), Platelet-derived growth factor receptor alpha (PDGFRA), Platelet-derived growth factor receptor beta (PDGFRB), Platelet-derived growth factor receptor-like (PDGFRL), Poliovirus receptor (PVR), Poliovirus receptor-related I (PVRL1), Semaphorin 4D (SEMA4D, CDt100), Solute earner family 11 member 2 (Slc11a2), Solute carrier family 11 member 2 (Slc11a2), Solute carrier family 11 member 2 (Slc11a2), Solute carrier family 16 member 1 (SLC16A1), Solute carrier family 26 member 2 (Slc26a2), Solute carrier family 26 member 2 (SLC26A2), Solute carrier family 40 member 1 (SLC40A1), Solute carrier family 7 member 2 (SLC7A2), Solute carrier family 7 member 4 (SLC7A4), Transferrin receptor (TFRC), Transferrin receptor (TFRC), Transient receptor potential cation channel (TRPV1), Transient receptor potential cation channel subfamily M member 8 (TRPM8), Tumor necrosis factor receptor superfamily member 10b, 10c, and 10d (TNFRSF10B, 10C, 10D), Two-pore calcium channel protein 2 (TPCN2), and Two-pore calcium channel protein 2 (TPCN2).

Additional experiments that may be performed according to embodiments of the invention include, for example, molecular imaging of L-selectin ligands and E-selectin in retinal and choroidal vasculature during LPS-induced uveitis (EIU); molecular imaging of choroidal neovascularization in an experimental model of age-related macular degeneration by targeting endothelial antigens, specific for neovascularization (e.g., αᵥβ₃ integrin) and molecules associated with CNV (e.g. ICAM-1); and molecular imaging of diabetic retinopathy in the STZ-induced model by targeting endothelial antigens, specific for endothelial injury during diabetes (e.g., ICAM-1).

Embodiments of the invention may be used in the diagnosis, staging, management, and/or treatment of any of a wide range of medical conditions, particularly those with one or more vascular, inflammatory, immune, and/or thrombotic components. Various categories of medical conditions include, for example, disorders of pain; of alterations in body temperature (e.g., fever): of nervous system dysfunction (e.g., syncope, myalgias, movement disorders, numbness, sensory loss, delirium, dementia, memory loss, sleep disorders); of the eyes, ears, nose, and throat; of circulatory and/or respiratory functions (e.g., dyspnea, pulmonary edema, cough, hemoptysis, hypertension, myocardial infarctions, hypoxia, cyanosis, cardiovascular collapse, congestive heart failure, edema, shock); of gastrointestinal function (e.g., dysphagia, diarrhea, constipation, GI bleeding, jaundice, ascites, indigestion, nausea, vomiting); of renal and urinary tract function (e.g., acidosis, alkalosis, fluid and electrolyte imbalances, azotemia, urinary abnormalities); of sexual function and reproduction (e.g., erectile dysfunction, menstrual disturbances, hirsutism, virilization, infertility, pregnancy associated disorders and standard measurements); of the skin (e.g., eczema, psoriasis, acne, rosacea, cutaneous infection, immunological skin diseases, photosensitivity); of the blood (e.g., hematology); of genes (e.g., genetic disorders); of drug response (e.g., adverse drug responses); and of nutrition (e.g., obesity, eating disorders, nutritional assessment). Other medical fields with which embodiments of the invention find utility include oncology (e.g., neoplasms, malignancies, angiogenesis, paraneoplasic syndromes, oncologic emergencies); hematology (e.g., anemia, hemoglobinopathies, megaloblastic anemias, hemolytic anemias, aplastic anemia, myelodysplasia, bone marrow failure, polycythemia vera, myloproliferative diseases, acute myeloid leukemia, chronic myeloid leukemia, lymphoid malignancies, plasma cell disorders, transfusion biology, transplants); hemostasis (e.g., disorders of coagulation and thrombosis, disorders of the platelet and vessel wall); and infectious diseases (e.g., sepsis, septic shock, fever of unknown origin, endocardidtis, bites, burns, osteomyelitis, abscesses, food poisoning, peliv inflammatory disease, bacterial (gram positive, gram negative, miscellaneous (nocardia, actimoyces, mixed), mycobacterial, spirochetal, rickettsial mycoplasma); chlamydia; viral (DNA, RNA), fungal and algal infections; protozoal and helminthic infections; endocrine diseases; nutritional diseases; and metabolic diseases.

Other medical conditions and/or fields with which embodiments of the invention find utility include those mentioned in Harrison's Principles of Internal Medicine, Kasper et al., ISBN 0071402357, McGraw-Hill Professional, 16th edition (2004) as well as those mentioned in Robbins Basic Pathology, Kumar, Cotran, and Robbins, eds., ISBN 1416025340, Elsevier, 7th edition (2005), both of which are incorporated herein by reference.

The subject matter of the following documents may be used in various embodiments of the invention; the texts of these documents are expressly incorporated herein by reference in their entirety for all purposes: (1) Hafezi-Moghadam, A., K. Noda, L. Almulki, E. F, tliaki, V. Poulaki, K. L. Thomas, T. Nakazawa, T. Hisatomi, J. W. Miller, and E. S. Gragoudas. 2006. VLA-4 Blockade Suppresses Endotoxin-Induced Uveitis: In Vivo Evidence for Functional Integrin Upregulation. FASEB J. FASEBJ/2006/063909: Published online Jan. 3, 2007 (pp. 1-11); (2) Hafezi-Moghadam, A., K. L. Thomas, A. J. Prorock, Y. Huo, and K. Ley. 2001. L-selectin shedding regulates leukocyte recruitment. J Exp Med 193:863-872; and (3) Hafezi-Moghadam, A., K. L. Thomas, and D. D. Wagner. 2006. ApoE-Deficiency Leads to a Progressive Age-Dependent Blood Brain Barrier Leakage. Am J. Physiol Cell Physiol (Jul. 26, 2006).

### EQUIVALENTS

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A minimally invasive method for the in vivo detection of one or more ligands on an intraluminal surface of a blood vessel, the method comprising:
(a) administering microparticles to a subject, wherein the microparticles have an average diameter less than a diameter of a blood vessel of the subject in which the microparticles travel, and wherein the microparticles have a surface to which one or more substances are conjugated, wherein the one or more substances interact with one or more ligands on an intraluminal surface of the blood vessel thereby inhibiting movement of the microparticles through the blood vessel; and
(b) detecting one or more of the administered microparticles in the blood vessel using a non-invasive detection device.

2. The method of claim 1, wherein the one or more substances conjugated to the surface of the microparticles bind to the one or more ligands on the intraluminal surface of the blood vessel.

3. The method of claim 1, wherein the one or more ligands comprise one or more native ligands.

4. The method of claim 1, wherein the one or more ligands comprise exogenous ligands.

5. The method of claim 1, wherein the one or more substances conjugated to the surface of the microparticles are covalently bound to the surface of the microparticles.

6. The method of claim 1, wherein the one or more substances conjugated to the surface of the microparticles are non-covalently associated with the surface of the microparticles.

7. The method of claim 1, wherein the microparticles are fluorescent microparticles and the detection device is a scanning laser ophthalmoscope.

8. The method of claim 1, wherein the one or more ligands on the intraluminal surface comprise an endothelial surface antigen, a leukocyte surface antigen, or both.

9. The method of claim 1, wherein the one or more ligands on the intraluminal surface comprise one or more members selected from the group consisting of a platelet antigen, a cell surface molecule, a micro-particle antigen, a protein, a lipid, a carbohydrate, a glycoprotein, a lipoprotein, a bacterial antigen, a viral antigen, a parasite antigen, and a cancer cell antigen.

10. The method of claim 1, wherein the one or more ligands accumulate on the intraluminal surface of the blood vessel.

11. The method of claim 1, comprising determining one or more parameters selected from the group consisting of a rolling parameter, a tethering parameter, and an adhesion parameter for one or more of the microparticles in the blood vessel, wherein the one or more parameters are indicative of the presence of one or more of the ligands on the intraluminal surface of the blood vessel.

12. The method of claim 11, wherein the one or more ligands on the intraluminal surface comprise an endothelial surface antigen, a leukocyte surface antigen, or both.

13. The method of claim 11, wherein the one or more parameters are indicative of inflammation in the blood vessel.

14. The method of claim 1, wherein the blood vessel is an ocular blood vessel.

15. The method of claim 14, wherein the blood vessel is located in retinal tissue, choroidal tissue, iris tissue, or conjunctival tissue.

16. The method of claim 1, further comprising identifying a sub-clinical manifestation of diabetic retinopathy based at least in part on the one or more microparticles detected in step (b).

17. The method of claim 1, further comprising identifying endothelial injury in a choroidal blood vessel based at least in part on the one or more microparticles detected in step (b).

18. The method of claim 17, comprising identifying endothelial injury in choriocapillaris during endotoxin-induced uveitis.

19.-71. (canceled)

72. A minimally invasive method for the in vivo determination of an endothelial condition associated with a disease, the method comprising:
(a) administering fluorescent microparticles to a subject, wherein the microparticles have an average diameter less than a diameter of a blood vessel of the subject in which the microparticles travel, and wherein the microparticles have a surface to which one or more substances are conjugated, wherein the one or more substances are capable of interacting with an endothelial marker of the disease, thereby inhibiting movement of the microparticles through the blood vessel; and
(b) detecting the administered fluorescent microparticles in one or more tissues of the subject using a scanning laser ophthalmoscope.

73.-79. (canceled)

80. A method of detecting ocular inflammation in a subject, the method comprising
(a) administering fluorescent microparticles to a subject, wherein one or more substances are conjugated to the surface of the microparticles, wherein the one or more substances interact with an endothelial surface antigen, a leukocyte surface antigen, or both, located on an intraluminal surface of a blood vessel in the subject, thereby inhibiting movement of the microparticles through the blood vessel; and
(b) determining one or more parameters selected from the group consisting of a rolling parameter, a tethering parameter, and an adhesion parameter, for one or more of the fluorescent microparticles in the blood vessel using a scanning laser ophthalmoscope, wherein the one or more parameters are indicative of whether the subject has ocular inflammation.

81.-108. (canceled)
